(19) United States
(12) Patent Application Publication
(10) Pub. No.: US 2024/0197691 A1
(43) Pub. Date: Jun. 20, 2024

(54) TACROLIMUS COMPOSITIONS AND METHODS OF USE

(71) Applicant: CN2 THERAPEUTICS, INC., Fort Lauderdale, FL (US)

(72) Inventor: FRANCIS E. O'DONNELL, Jr., Longboat Key, FL (US)

(21) Appl. No.: 18/066,476

(22) Filed: Dec. 15, 2022

(51) Int. Cl. A61K 31/436; A61F 9/00; A61K 9/00; A61K 41/17; A61K 47/02; A61K 47/10

(52) U.S. Cl. CPC A61K 31/436; A61F 9/0008; A61K 9/0048; A61K 41/17; A61K 47/02; A61K 47/10

(57) ABSTRACT

The subject invention pertains to tacrolimus compositions and a method for the treatment of symptoms of allergic conjunctivitis or keratoconjunctivitis, including, for example, ocular redness and ocular itchiness. The composition is preservative free and can be dispensed in a multidose container.

### BACKGROUND OF THE INVENTION

Allergic conjunctivitis or keratoconjunctivitis leads to moderate to severe allergic eye symptoms and sight-threatening complications. Patients typically present with eye itching, tearing, discharge, irritation, redness, blepharospasm, and photophobia. Allergic conjunctivitis or keratoconjunctivitis can also comprise of chronic, non-seasonal allergic diseases with active flare-ups in addition to seasonal allergic diseases.

Current management of allergic conjunctivitis typically progresses from a topical antihistamine drop to topical steroids if the ocular allergy cannot be controlled. Topical steroids can place patients at greater risk for infection, cataract, and glaucoma. The topical steroids can be effective in management of flare-ups, yet the side effects are not ideal for long-term management. The main cause of vision loss in atopic keratoconjunctivitis (AKC) is chronic use of topical steroids. Alternatives to topical steroids include cyclosporin; several formulations of cyclosporin are approved for treating dry eyes, and allergic conjunctivitis, specifically vernal keratoconjunctivitis (VKC) but failed in AKC trials.

TALYMUS ophthalmic suspension containing 0.1% tacrolimus has been approved formulation in Japan since 2008 for AKC (atopic keratoconjunctivitis) and VKC (vernal keratoconjunctivitis) forms of allergic keratoconjunctivitis. Numerous corporate and investigator sponsored clinical trials of approved product in Japan establish the efficacy in severe allergic KC due to vernal or atopic diseases. However, unacceptable side effects exist for use of TALYMUS for the US market: 40% incidence of eye burning and irritation.

Therefore, there remains a need for a topical preparation that is effective in the relief of ocular itching and redness owing to an allergic eye condition and that is safer to use than topical steroids.

### BRIEF SUMMARY OF THE INVENTION

The present invention provides tacrolimus compositions and a method for the treatment of symptoms of allergic conjunctivitis or keratoconjunctivitis, including, for example, ocular redness and ocular itchiness. In certain embodiments, the composition is preservative free and can be dispensed in a multidose container. In certain embodiments, the tacrolimus composition or tacrolimus powder used to create the composition is sterilized by gamma irradiation. In certain embodiments, the gamma irradiation does not degrade the tacrolimus molecules.

In certain embodiments, the tacrolimus compositions comprise about 0.01% to about 1% tacrolimus, preferably about 0.05% tacrolimus to about 0.5% tacrolimus, or, most preferably, about 0.1% tacrolimus. In certain embodiments, the pH of the composition is about 6 to about 9, preferably, about 7 to about 8, or, most preferably, about 7.35 to about 7.45 (i.e., physiologic pH).

In certain embodiments, the composition is micronized with a particle size of about 0.1 m to about 10 m. In certain embodiments, the compositions is administered in a multidose container that obviates the need for preservatives. In certain embodiments, the composition has a viscosity of about 6.7 centipoise (cP) to about 25 cP, preferably about 10 cp to about 24 cP, most preferably, about 23.4 cP, encouraging a longer residence time on the ocular surface when compared to composition with a lower viscosity. In certain embodiments, the contact angle of the tacrolimus compositions is about 68.0° to about 79.0°, about 70.0° to about 75.0°, about 73.0° to about 74.0°, or about 73.2° to about 73.5°. The tacrolimus composition has better wettability on the hydrophobic surface of parafilm when compared to water (97.9° and 99.9°).

In certain embodiments, the subject composition and methods do not affect the innate immune system but provide a suppression of the adaptive immune system that is responsible for the release of cytokines that result in mobilization of inflammatory cells.

### BRIEF DESCRIPTION OF THE DRAWINGS

FIG. 1. Molecular pathways of cytokine release

FIG. 2. Use of tacrolimus in various grades of allergic conjunctivitis

FIG. 3. Contact angle measurements of water (20 μL) on parafilm at an ambient temperature or 97.9° and 99.9°.

FIG. 4. Contact angle measurements of tacrolimus suspension (20 μL) on parafilm at an ambient temperature or 73.5° and 73.2°.

FIGS. 5A-5C. Tacrolimus Suspension Container and Dispenser (FIG. 5A), Liner Cap (FIG. 5B), and filter protection valve (FIG. 5C).

FIG. 6. The total dissolution for all replicates (and resulting average) for prototypes P20 (micronized, avg. shown in red) and P24 (microfluidized, avg. shown in blue), as measured in the receptor fluid of in vitro release experiments

FIG. 7. The percent Tacrolimus released for each replicate (and the resulting average) for prototypes P20 (micronized, avg. shown in red) and P24 (microfluidized, avg. shown in blue), based on the total available amount of Tacrolimus in the suspension sample contained in the Float-A-Lyzer device.

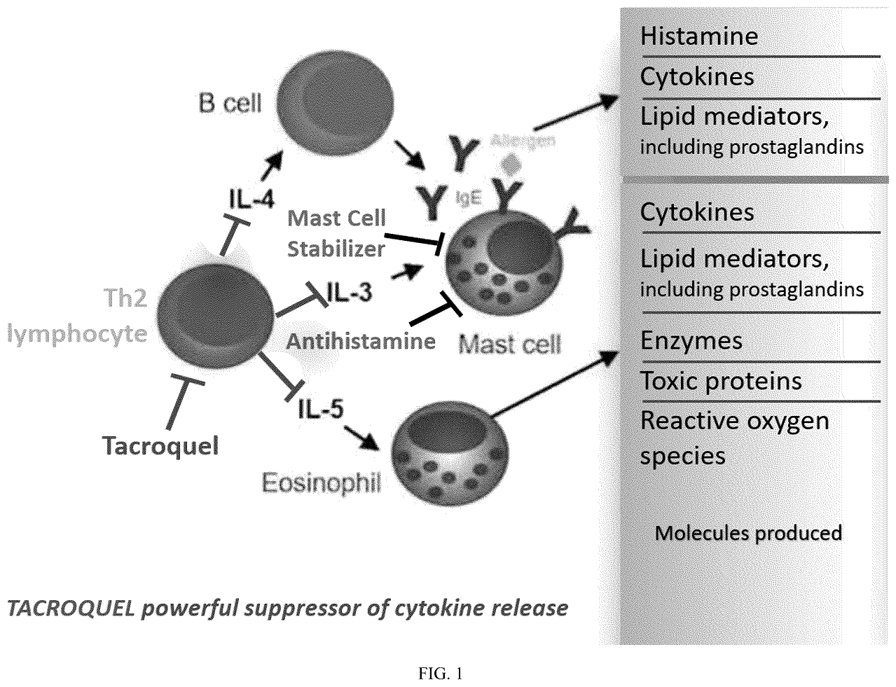

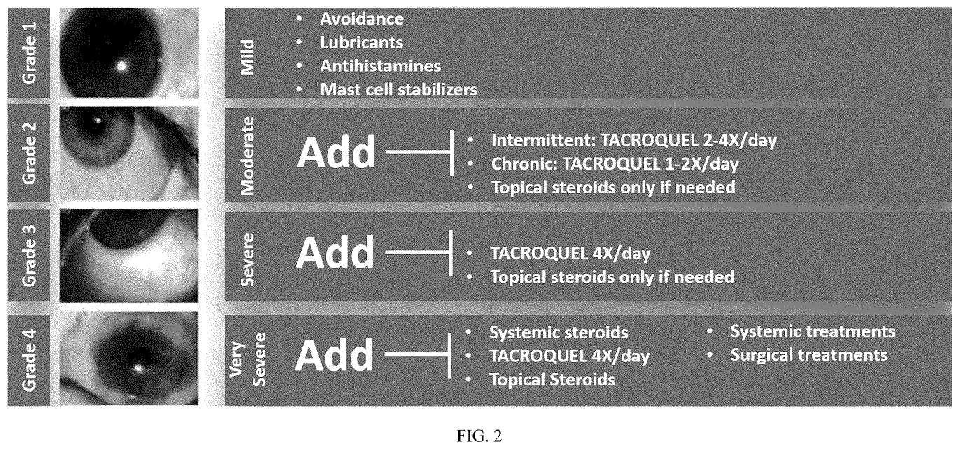

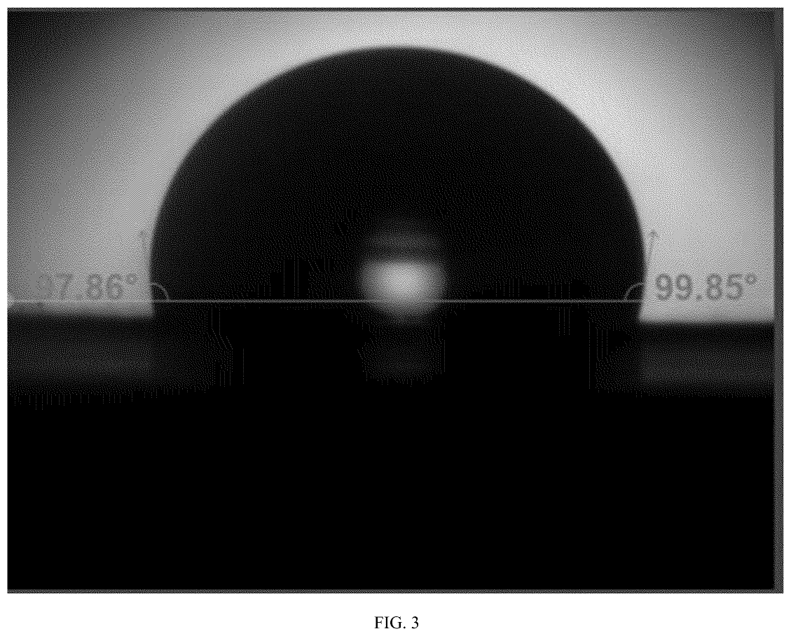

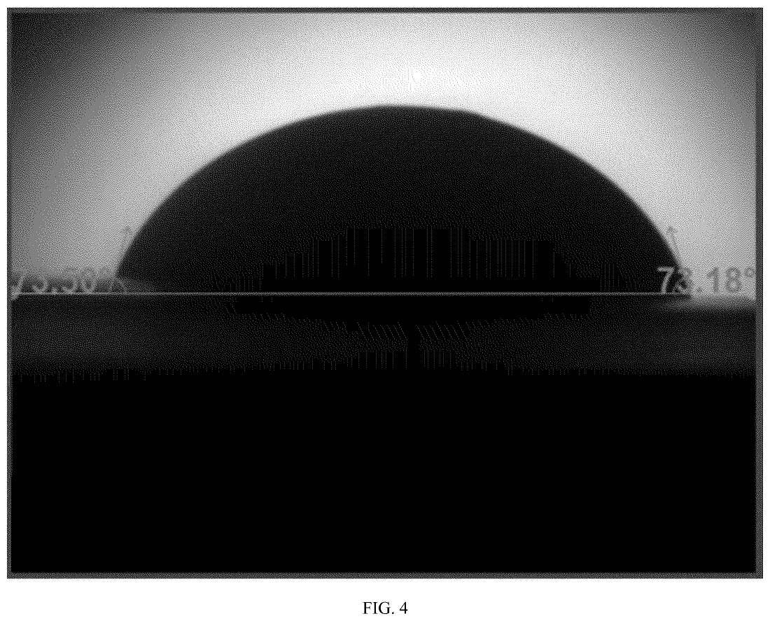

### DETAILED DISCLOSURE OF THE INVENTION

### Selected Definitions

As used herein, the singular forms “a,” “an,” and “the” are intended to include the plural forms as well, unless the context clearly indicates otherwise. Further, to the extent that the terms “including,” “includes,” “having,” “has,” “with,” or variants thereof are used in either the detailed description and/or the claims, such terms are intended to be inclusive in a manner similar to the term “comprising.” The transitional terms/phrases (and any grammatical variations thereof) “comprising,” “comprises,” “comprise,” include the phrases “consisting essentially of,” “consists essentially of,” “consisting,” and “consists.”

The phrases “consisting essentially of” or “consists essentially of” indicate that the claim encompasses embodiments containing the specified materials or steps and those that do not materially affect the basic and novel characteristic(s) of the claim.

The term “about” means within an acceptable error range for the particular value as determined by one of ordinary skill in the art, which will depend in part on how the value is measured or determined, i.e., the limitations of the measurement system. Where particular values are described in the application and claims, unless otherwise stated the term “about” meaning within an acceptable error range for the particular value should be assumed.

In the present disclosure, ranges are stated in shorthand, to avoid having to set out at length and describe each and every value within the range. Any appropriate value within the range can be selected, where appropriate, as the upper value, lower value, or the terminus of the range. For example, a range of 1-10 represents the terminal values of 1 and 10, as well as the intermediate values of 2, 3, 4, 5, 6, 7, 8, 9, and all intermediate ranges encompassed within 1-10, such as 2-5, 2-8, and 7-10. Also, when ranges are used herein, combinations and sub-combinations of ranges (e.g., subranges within the disclosed range) and specific embodiments therein are intended to be explicitly included.

In certain embodiments of the invention a subject is a mammal. Non-limiting examples of a mammal treatable according to the methods of the current invention include mouse, rat, dog, guinea pig, cow, horse, cat, rabbit, pig, monkey, ape, chimpanzee, and human. Additional examples of mammals treatable with the methods of the current invention are well known to a person of ordinary skill in the art and such embodiments are within the purview of the current invention.

For the purposes of this invention the terms “treatment, treating, treat” or equivalents of these terms refer to healing, alleviating, relieving, altering, remedying, ameliorating, improving, or affecting the condition or the symptoms of a subject suffering with a disease or condition, for example, allergic conjunctivitis. The subject to be treated can be suffering from or at risk of developing the disorder or condition, for example, allergic conjunctivitis. When provided therapeutically, the compound can be provided before the onset of a symptom. The therapeutic administration of the substance serves to attenuate any actual symptom.

For the purposes of this invention, the terms “preventing, preventive, prophylactic” or equivalents of these terms are indicate that the compounds of the subject invention are provided in advance of any disease symptoms and are a separate aspect of the invention (i.e., an aspect of the invention that is distinct from aspects related to the terms “treatment, treating, treat” or equivalents of these terms which refer to healing, alleviating, relieving, altering, remedying, ameliorating, improving, or affecting the condition or the symptoms of a subject suffering from allergic conjunctivitis or symptoms thereof). The prophylactic administration of the compounds of the subject invention serves to prevent, reduce the likelihood, or attenuate one or more subsequent symptoms or condition.

By “therapeutically effective dose,” “therapeutically effective amount”, or “effective amount” is intended to be an amount of a compounds of the subject invention disclosed herein that, when administered to a subject, decreases the number or severity of symptoms or inhibits or eliminates the progression or initiation of allergic conjunctivitis or reduces any increase in symptoms, or improve the clinical course of the disease as compared to untreated subjects. “Positive therapeutic response” refers to, for example, improving the condition of at least one of the symptoms of allergic conjunctivitis.

An effective amount of the therapeutic agent is determined based on the intended goal. The term “unit dose” refers to a physically discrete unit suitable for use in a subject, each unit containing a predetermined quantity of the therapeutic composition calculated to produce the desired response in association with its administration, i.e., the appropriate route and treatment regimen. The quantity to be administered, both according to number of treatments and unit dose, depends on the subject to be treated, the state of the subject and the protection desired. Precise amounts of the therapeutic composition also depend on the judgment of the practitioner and are peculiar to each individual. Generally, the dosage of the compounds of the subject invention will vary depending upon such factors as the patient's age, weight, height, sex, general medical condition and previous medical history.

In some embodiments of the invention, the method comprises administration of multiple doses of the compounds of the subject invention. The method may comprise administration of 1, 2, 3, 4, 5, 6, 7, 8, 9, 10, 15, 20, 25, 30, 35, 40, 50, 75, 100, 150, 200, 250, 300, 350, 400, 450, 500, 600, 700, 800, 900, 1000 or more therapeutically effective doses of a composition comprising the compounds of the subject invention as described herein. In some embodiments, doses are administered over the course of 1 day, 2 days, 3 days, 4 days, 5 days, 6 days, 7 days, 10 days, 14 days, 21 days, 28 days, 30 days, 2 months, 3 months, 6 months, 1 year, 2 years, 3 years, 4 years, 5 years, 6 years, 7 years, 8 years, 9 years, 10 years, or more than 10 years. The frequency and duration of administration of multiple doses of the compositions is such as to inhibit or delay the initiation of allergic conjunctivitis or symptoms thereof. Moreover, treatment of a subject with a therapeutically effective amount of the compounds of the invention can include a single treatment or can include a series of treatments. It will also be appreciated that the effective dosage of a compound used for treatment may increase or decrease over the course of a particular treatment. Changes in dosage may result and become apparent from the results of diagnostic methods for detecting allergic conjunctivitis or symptoms thereof known in the art. In some embodiments of the invention, the method comprises administration of the compounds at a single time per day or several times per day, including but not limiting to 2 times per day, 3 times per day, and 4 times per day.

As used herein, the term “drop” refers to a unit of measure of volume, which is equal to the amount dispensed as one drop from a dropper or drip chamber. In certain embodiments, a drop can contain about 20 μl to about 100 μl, about 30 μl to 70 μl, or about 50 μl of liquid.

As used herein, the terms “micronized” or “micronization” refer to independent processes of decreasing the size of a particle to that of the micrometer or nanometer scale.

### Compounds

In preferred embodiments, the compositions and methods according to the subject invention utilize tacrolimus. Tacrolimus (TAC), also known as FK-506, is a complex 23-membered macrolide lactone immunosuppressive drug. TAC has two kinds of conformational structures—cis- or trans at the amide bond—and isomerizes in polar solvents by undergoing a hydration reaction to a diol at the amide bond, that can convert into the C-10 epimer. An equilibrium with all three co-existing forms according to formula (I) (TAC1), formula (II) (TAC2), and formula (III) (TAC3) of TAC exists:

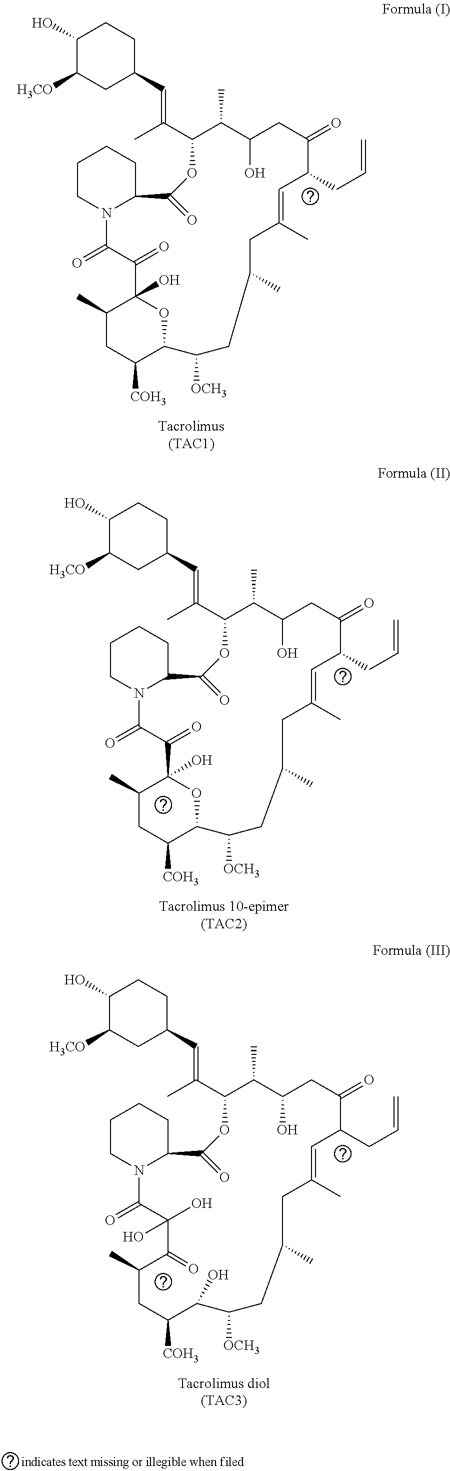

Tacrolimus may be added to compositions at concentrations of about 0.0001 to about 5% by weight (wt %), preferably about 0.01 to about 0.5 wt %, and most preferably about 0.1 wt %. In another embodiment, tacrolimus can be in combination with an acceptable carrier and/or excipient, in that tacrolimus may be presented at concentrations of about 0.0001 to about 5% (v/v), preferably, about 0.01 to about 0.5% (v/v), more preferably, about 0.05 to about 0.25% (v/v), or, most preferably about 0.1% (v/v).

In certain embodiments, the particle size distributions of tacrolimus in the composition can be about 0.01 μm to about 100 μm, about 0.1 μm to about 10 μm, about 1 μm to about 7 μm or about 1.72 μm, about 2.94 μm, about 6.80 μm, about 0.28 μm, about 0.78 μm, about 1.68 μm and span of about 0.1 to about 10, about 1 to about 5, about 1.78 or about 1.73.

TABLE 1
Formulation of Ophthalmic Tacrolimus 0.1%

| Components | Weight percent |
|---|---|
| Tacrolimus | 0.10% |
| Glycerin | 2.12% |
| Sodium Phosphate Monobasic Monohydrate | 0.11% |
| Dibasic Sodium Phosphate Heptahydrate | 0.33% |
| Dispersant: PEG 40 Stearate | 0.10% |
| Viscosifier: HPMC 2910 (4000) | 0.45% |
| WFI | QS |

TABLE 2
Characterization of Ophthalmic Tacrolimus 0.1%
Characterization

| Osmolarity (mOsm/Kg) | | | 300 |
|---|---|---|---|
| | pH | | 7.10 |
| | Viscosity (cP), 25° C., SR: 50 1/s | | 23.4 |
| | PSD | D10 (μm) | 1.72 |
| | | D50 (μm) | 2.67 |
| | | D90 (μm) | 4.44 |
| | | Span | 1.02 |

| Assay (mg/g) | | 1.03 |
|---|---|---|
| | Total Impurity (%) | 0.62% |

In certain embodiments, the compositions are preferably administered to the eye, including, for example, ophthalmic administration, conjunctival administration, intracorneal administration, intraocular administration, intravitreal administration, or retrobulbar administration.

In one embodiment, the composition is a, syrup, emulsion, or liquid suspension containing a desired substance.

The subject composition can further comprise one or more pharmaceutically acceptable carriers, and/or excipients, and can be formulated into preparations, for example, semi-solid, liquid, or gaseous forms, such as ointments, solutions, suppositories, and injections.

The term “pharmaceutically acceptable” as used herein means compatible with the other ingredients of a pharmaceutical composition and not deleterious to the recipient thereof.

Carriers and/or excipients according the subject invention can include any and all solvents, diluents, buffers (such as, e.g., neutral buffered saline, phosphate buffered saline, or optionally Tris-HCl, acetate or phosphate buffers), oil-in-water or water-in-oil emulsions, aqueous compositions with or without inclusion of organic co-solvents suitable for use, e.g., IV solubilizers (e.g., Polysorbate 65, Polysorbate 80), colloids, dispersion media, vehicles, fillers, chelating agents (e.g., EDTA or glutathione), amino acids (e.g., glycine), proteins, disintegrants, binders, lubricants (e.g., glycerin, polyethylene glycol (PEG)), osmolality adjuster, emollient, wetting agents, emulsifiers, sweeteners, colorants, flavorings, aromatizers, thickeners (e.g. carbomer, gelatin, or sodium alginate), coatings, tonicity controlling agents, absorption delaying agents, adjuvants, bulking agents (e.g., lactose, mannitol) and the like. The use of carriers and/or excipients in the field of drugs and supplements is well known. Except for any conventional media or agent that is incompatible with the target health-promoting substance or with the composition, carrier or excipient use in the subject compositions may be contemplated. In certain embodiments, the compositions can comprise a polymer, such as, for example, hydroxypropyl methylcellulose (HPMC)-606, a salt, such as, for example, sodium phosphate monobasic, sodium chloride, sodium borate, and an acid, such as, for example, boric acid or hydrochloric acid.

In certain embodiments, the compositions can be formulated as about 0.1% to about 10%, about 1% to about 10%, or about, 2.12% emollient (e.g., glycerin), about 0.01% to about 10% or about 0.1% to about 1% buffer (e.g., sodium phosphate monobasic monohydrate, dibasic sodium phosphate heptahydrate), about 0.01% to about 10%, about 0.01% to about 1%, or about 0.1% dispersant (e.g., PEG 40 stearate), about 0.01% to about 10%, about 0.1% to about 5%, or about 0.45% of a viscosifier (e.g., HPMC 2910 (4000)), and the balance Water for Injection (WFI).

In one embodiment, the compositions of the subject invention can be formulated for administration, for example, as a solution or suspension. The solution or suspension can comprise suitable non-toxic, parenterally-acceptable diluents or solvents, such as mannitol, 1,3-butanediol, water, Ringer's solution, or isotonic sodium chloride solution, or suitable dispersing or wetting and suspending agents, such as sterile, non-irritant, fixed oils, including synthetic mono- or diglycerides, and fatty acids, including oleic acid. One illustrative example of a carrier includes a mixture of 10% USP ethanol, 40% USP propylene glycol or polyethylene glycol 600 and the balance USP WFI. Other illustrative carriers use include 10% USP ethanol and USP WFI; 0.01-0.1% triethanolamine in USP WFI; or 0.01-0.2% dipalmitoyl diphosphatidylcholine in USP WFI; and 1-10% squalene or parenteral vegetable oil-in-water emulsion. Water or saline solutions and aqueous dextrose and glycerol solutions may be preferably employed as carriers, particularly for solutions. Illustrative examples of carriers for use include phosphate buffered saline (PBS) solution, 5% dextrose in WFI and 0.01-0.1% triethanolamine in 5% dextrose or 0.9% sodium chloride in USP WFI, or a 1 to 2 or 1 to 4 mixture of 10% USP ethanol, 40% propylene glycol and the balance an acceptable isotonic solution such as 5% dextrose or 0.9% sodium chloride; or 0.01-0.2% dipalmitoyl diphosphatidylcholine in USP WFI and 1 to 10% squalene or parenteral vegetable oil-in-water emulsions.

In certain embodiments, the composition can be irradiated before administration to a subject. For example, at least one component of the composition, such as, for example, tacrolimus, can undergo gamma irradiation before the final composition is created or the final composition can undergo gamma irradiation. In certain embodiments, the composition can be irradiated at a dose ranging from about 1 kilo-Gray (kGy) to about 100 kGy, preferably about 10 kGy to about 75 kGy, most preferably about 15 kGy to about 40 kGy.

In certain embodiments, the compositions of the subject invention have a stable pH, osmolality, particle size distribution (PSD), tacrolimus concentration (assay), total impurities, viscosities, or any combination thereof at a temperature of about 5° C. to about 40° C. condition for at least about 1 day, about 3 days, about 7 days, about 2 weeks, about 4 weeks, about 6 weeks, about 8 weeks, about 2 months, about 12 weeks, or about three months.

In certain embodiments, the compositions of the subject invention can be synthesized using a microfluidization process. In certain embodiments, the microfluidation process can comprise mixing tacrolimus into a first solution, such as, for example, solution I; microfluidizing the tacrolimus and solution mixture with compressed air; and diluting the tacrolimus and solution suspension with a second solution, such as, for example, solution II or the second solution and additional first solution, wherein solution I comprises sodium phosphate monobasic monohydrate, dibasic sodium phosphate heptahydrate, and a dispersant; and solution II comprises a viscosifier, glycerin, sodium phosphate monobasic monohydrate, dibasic sodium phosphate heptahydrate, and a dispersant. In certain embodiments, the microfluidizing step comprises at 2, 3, 4, 5, 6, 7, 8, 9, 10, about 15, about 20, about 25, about 30, about 35, about 40, about 45, or about 50 passes of the tacrolimus suspension in the first solution through a microfluidization instrument.

In certain embodiments, the compositions of the subject invention can be synthesized using a micronization process by mixing tacrolimus into Solution I; and diluting the tacrolimus and solution I suspension with solution II, wherein solution I comprises sodium phosphate monobasic monohydrate, dibasic sodium phosphate heptahydrate, and a dispersant; and solution II comprises a viscosifier, glycerin, sodium phosphate monobasic monohydrate, dibasic sodium phosphate heptahydrate, and a dispersant.

### Method of Use of Subject Compositions

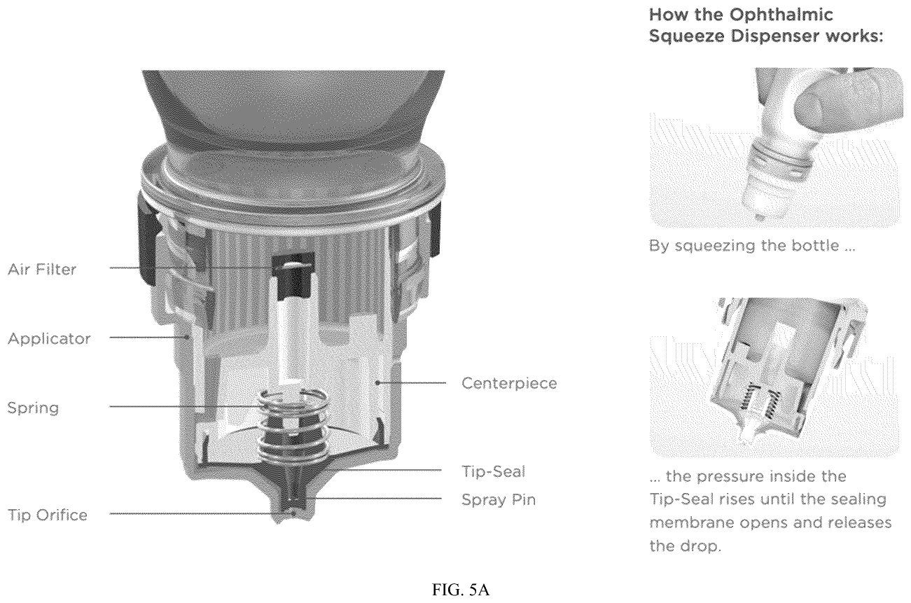

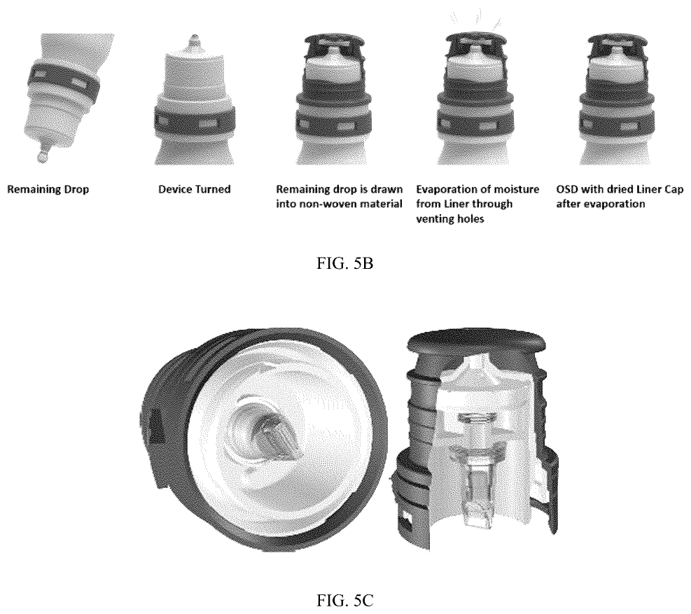

In certain embodiments, a delivery system, such as, for example, Aptar Delivery System (U.S. Pat. Nos. 9,815,609, 9,833,356, and 11,059,639) can be used to administer the subject compositions using a multi-dose dispenser with unique tip technology that eliminates need for preservative (FIG. 5A). The delivery system can comprise a thirty-day supply of for administration of at least 1, 2, 3, or, most preferably 4 times per day.

In certain embodiments, the delivery system can be designed for preservative free formulations in which the delivery system comprises a tip seal and filter. The tip seal mechanism closes the orifice immediately when releasing the pressure, thus preventing contamination through the tip. The delivery system is suitable for sensitive formulations due to metal free fluid path. In certain embodiments, the air required to compensate the volume loss in the delivery system after the actuation is filtered through a membrane filter to reduce or eliminate impurities or contaminants (e.g., bacteria), such as, for example a polytetrafluoroethylene (PTFE) multilayer membrane. In certain embodiments, the delivery system can comprise a cap (e.g., liner cap) featuring a material (e.g., a fabric made of a nonwoven polypropylene/polyethylene (PP/PE) blend) intended to wipe off the residual drop with each use (FIG. 5B). This cap reduces or eliminates formulation crystallization. In certain embodiments, the delivery system can comprise a filter protection valve (FIG. 5C). The Filter protection valve can be a one-way valve separating media and filter membrane, which allows the bottle venting by a one-way airflow.

In certain embodiments, the compositions comprising tacrolimus can be administered to a subject to inhibit or delay the onset of ocular redness or ocular itchiness caused by allergic conjunctivitis by administration to the eye. In some embodiments, treating the subject with the composition disclosed herein can reduce or eliminate ocular redness or ocular itchiness. In some embodiments, doses are administered over the course of about 1 day, about 2 days, about 7 days, about 14 days, about 21 days, about 28 days, about 30 days, about 2 months, about 3 months, about 6 months, about 9 months, about 1 year, about 2 years, about 3 years, about 4 years, about 5 years, about 6 years, about 7 years, about 8 years, about 9 years, about 10 years, or more than 10 years.

In certain embodiments, the subject composition can be delivered to the subject via a topical route, formulated as solutions, suspensions, emulsions, gels, ointments, pastes, etc. In preferred embodiments, the composition can be a liquid solution to be applied to the eye of a subject (i.e., ophthalmic administration). In certain embodiments, at least 1, 2, 3, 4, 5, 6, 7, 8, 9, or 10 drops can be administered to a subject. In certain embodiments, one drop can have a volume of about 20 μL to about 100 μL, about 25 μL to about 75 μL, or about 25 μL to about 45 L. In certain embodiments, each drop can comprise a dose of tacrolimus of about 1 mg to about 100 mg, about 5 mg to about 75 mg, about 30 mg to about 50 mg, or about 35 mg.

In certain embodiments, the composition can be administered at any time in a day. In certain embodiments, the composition can be administered to a subject once, twice, thrice, four-times, or more times per day. In certain embodiments, the composition can be administered every day, every two days, every three days, or every four days. In certain embodiments, each administration can occur at a consistent time period, volume, and concentration. In certain embodiments, the time period between administrations can increase or decrease during the treatment period. In certain embodiments, the volume of the administrations can increase or decrease during the treatment period. In certain embodiments, the concentration of the compositions administered can increase or decrease during the treatment period.

In certain embodiments, the treatment period can occur for at least about 1 week, about 1 month, about 3 months, about 6 months, about 9 months, about one year, about two years, about 3 years, about four years, about five years, about six years, about seven years, about eight years, or about ten years. In certain embodiments, the composition can be administered 6 months to about 1 year, 18 months to 2 years, 1 year to 2 years, 16 to 32 months, 24 to 48 months, 32 to 48 months, 32 to 52 months, 48 to 52 months, 48 to 64 months, 52 to 64 months, 52 to 72 months, 64 to 72 months, 64 to 80 months, 72 to 80 months, 72 to 88 months, 80 to 88 months, 80 to 96 months, 88 to 96 months, and 96 to 104 months. Suitable periods of administration also include 1, 2, 3, 4, 5, 6, 7, 8, 9, 10, 11, 12, 13, 14, 15, 16, 17, 18, 19, 20, 24, 28, 30, 32, 36, 42, 48, and 56 months. Generally administration of the composition should be continued as long as clinically significant ocular redness or ocular itchiness is observed.

In some embodiments, administration of an tacrolimus composition is not continuous and can be stopped for one or more periods of time, followed by one or more periods of time where administration resumes. Suitable periods where administration stops include 1 to 9 months, 5 to 16 months, 9 to 16 months, 16 to 24 months, 16 to 32 months, 24 to 32 months, 24 to 48 months, 32 to 48 months, 32 to 52 months, 48 to 52 months, 48 to 64 months, 52 to 64 months, 52 to 72 months, 64 to 72 months, 64 to 80 months, 72 to 80 months, 72 to 88 months, 80 to 88 months, 80 to 96 months, 88 to 96 months, and 96 to 100 months. Suitable periods where administration stops also include 1, 2, 3, 4, 5, 6, 7, 8, 9, 10, 11, 12, 13, 14, 15, 16, 17, 18, 19, 20, 24, 25, 30, 32, 35, 40, 45, 48 50, 52, 55, 60, 64, 65, 68, 70, 72, 75, 80, 85, 88 90, 95, 96, and 100 months.

In certain embodiments, the subject tacrolimus compositions can be used in combination with other active agents known to be useful for reducing or eliminating ocular redness or ocular itchiness cause by allergic conjunctivitis.

In certain embodiments, the subject composition can be used treat anterior segment inflammatory disorders or symptoms thereof including, for example, chronic anterior uveitis, graft versus host disease, ocular cicatricial pemphigoid, Stevens-Johnson syndrome, superior limbic keratoconjunctivitis, adenoviral keratitis, phlyctenular keratoconjunctivitis, penetrating keratoplasty, and scleritis.

### Materials and Methods

### Microfluidization and Micronization:

A series of formulations were prepared in 20 mM Phosphate Buffer, pH 7.0 with 0.1% w/w of surfactant. Prototypes prepared via the microfluidization process were based on a two-solution process. Solution I (Table 4), the microfluidization solution, was a highly concentrated solution of Tacrolimus (10 mg/g). The higher the concentration of Tacrolimus, the greater the number of collisions during the microfluidization process, and thus the greater the potential for particle size reduction. The presence of a viscosifier was intentionally omitted from Solution I, as the presence of a polymer would likely inhibit the particle-to-particle interactions, and the high shear from the microfluidization process would likely result in chain cleavage of the polymer, thus reducing the viscosity. The process design would require a dilution from 10 mg/g to 1 mg/g, using Solution II (Table 5), which would be a polymer containing vehicle.

In the microfluidization experiments three different surfactants were evaluated and were present in both Solution I and Solution II, at equal concentrations. The surfactants evaluated were Poloxamer 188, Polyoxyl 40 Stearate, and Polysorbate 80. The concentration of the surfactant was maintained at 0.1% w/w for each surfactant evaluated. As part of the microfluidization experiments, the addition of glycerin (serves as an osmolality adjuster, and as an emollient) to solution I was also evaluated for its effect on particle size reduction and impurities.

The microfluidization was performed using Microfluidizer Triad Model 110Y, and compressed air. A Z-channel configuration was used, and the formulations were processed with a series of 20 passes through the microfluidizer, with periodic sampling after passes. Initial microfluidization experiments were performed without temperature control. Particle size of the samplings was measured by laser light scattering, and the impurities were measured by ultra-high-pressure liquid chromatography. The effects of annealing overnight at 40° C. after microfluidization on the particles' stability was also evaluated. The purpose of annealing is to facilitate the dissipation of energy after the microfluidization process. The high energy state may result in a repulsion or aggregation of the API particles. Thus, annealing would allow the suspension to readily reach a more thermodynamically stable state of the particle-to-particle interaction.

### Franz Cell Setup

The cornea from six bovine eyes were excised and stored in a glutathione buffer at 2-8° C. (for less than 24 hours), prior to use for the corneal permeability experiments. The receptor fluid was prepared (see Table 3 for composition of receptor fluid), pH was adjusted to 7.0, and filtered (0.22 μm PVDF).

TABLE 3
Composition of receptor fluid

| Component | w/w % | (g) |
|---|---|---|
| Monosodium phosphate monohydrate | 0.11% | 0.106 |
| Disodium phosphate heptahydrate | 0.33% | 0.331 |
| Hydroxypropyl-β-cyclodextrin | 5.00% | 5.00 |
| Sodium Chloride | 0.6% | 0.60 |
| Water | QS | 94.56 |

The Franz cells (each containing a small flea-sized magnet) were each filled with ˜5 mL of receptor fluid, and topped off such that a droplet formed a convex meniscus above the site of cornea placement at the ball joint. The weight of receptor fluid transferred into each Franz cell was obtained.

Prior to placement of the cornea onto the Franz cell, the thickness of the cornea was measured using a digital Vernier caliper. The corneas were carefully put into place using forceps, ensuring that there were no bubbles at the interface of the receptor fluid and cornea. The donor chamber was connected to the ball joint above the cornea and clamped for each cell. Using a Drummond pipette, 200 μL of suspension was inserted into the Franz cell donor chamber, and the weights of suspension recorded (weights obtained by difference). Each suspension was evaluated in triplicate (i.e. three Franz cells per suspension). The Franz cells were connected to the heater/chiller circulator set to 37° C., and the initial time (TO) was noted.

### Sampling Method And Analysis Of Receptor Fluid

At designated time intervals, 300 μL of receptor fluid was removed from the side-arm of the Franz Cell using a Drummond pipette. Fresh receptor fluid was replaced into the Franz cell to match the volume removed. Based on the weight of the sample volume, 10% of the weight was acidified acetonitrile was added at 10% of the sample weight (acidified diluent was added to establish Equilibrium among the three tacrolimus chromatography peaks). The samples were then analyzed by ultra-high pressure liquid chromatography (UHPLC).

### Extraction of Cornea

Upon completion of the experiment, the Franz cell was carefully dismantled, and the cornea were gently wetted with distilled water and blotted with lab tissue. The thickness of the corneas was remeasured with the digital vernier calipers. Using a hole punch (¼″ diameter), each cornea was cutout, and transferred into a tared vial and weighed. Approximately 1 g of diluent (acetonitrile: water, 1:1, v/v) was added to each cornea, and stored at 5° C. prior to workup. Three bead ceramic bead were added to each vial, and the vials were placed into a bead miller homogenizer for 5 minutes. The resulting homogenate was centrifuged at 13k rpm for 2 minutes. The supernatant was removed and filtered (0.45 μm PTFE), and then analyzed by UHPLC.

### Preparation of Prototypes via Procedure I: Microfluidization Process

Suspensions prepared via the microfluidization process were prepared by first dispersing the Tacrolimus (10 mg/g) into Solution I by overhead mixing using a Scilogex mixer. The resulting suspension was microfluidized, with cooling, for a specified number of passes. The dilution scheme was roughly 1:9 using one part of the microfluidized suspension prepared with Solution I, to nine parts of the polymer (and glycerin) containing vehicle Solution. Prior to the dilution, the microfluidized suspension was assayed for the precise Tacrolimus concentration (which typically was <10 mg/g, due to losses during the microfluidization process), and was subsequently diluted with Solution II (and using a portion of Solution I without Tacrolimus) to achieve the target final concentration of Tacrolimus (0.1% w/w)), HPMC (0.5% w/w).

TABLE 4
Solution I composition

| Component | Weight (%) |
|---|---|
| Tacrolimus | 1.00% |
| Sodium Phosphate Monobasic Monohydrate | 0.13% |
| Dibasic Sodium Phosphate Heptahydrate | 0.28% |
| Dispersant (e.g. Polyoxyl 40 Stearate) | 0.10% |
| Water | QS |

### Preparation of Prototypes vis Procedure II: Unprocessed Tacrolimus

Suspensions prepared using unprocessed (micronized) Tacrolimus were prepared by first dispersing the Tacrolimus (4 mg/g) into Solution I by overhead mixing using a Scilogex mixer. The resulting suspension was diluted with Solution II in a 1:3 dilution scheme, using one part of the Tacrolimus suspension prepared using Solution I, to three parts of Solution II.

TABLE 5
Solution II Composition

| Component | Weight (%) |
|---|---|
| Viscosifier: (e.g. HPMC) | 0.66% |
| Glycerin | 2.42% |
| Sodium Phosphate Monobasic Monohydrate | 0.13% |
| Dibasic Sodium Phosphate Heptahydrate | 0.28% |
| Dispersant (e.g. Polyoxyl 40 Stearate) | 0.10% |
| Water | OS |

All patents, patent applications, provisional applications, and publications referred to or cited herein are incorporated by reference in their entirety, including all figures and tables, to the extent they are not inconsistent with the explicit teachings of this specification.

Following are examples that illustrate procedures for practicing the invention. These examples should not be construed as limiting. All percentages are by weight and all solvent mixture proportions are by volume unless otherwise noted.

### Example 1-Pre-Formulation Stability and Solubility of Tacrolimus

The stability and solubility of Tacrolimus suspensions (2 mg/g) were evaluated in three buffer systems: citrate acetate, and phosphate; citrate and acetate were evaluated at pH 5.0, 5.5 and 6.0, while phosphate was tested at pH 6.0, 6.5, 6.8, 7.0, 7.2 and 7.4 with all samples being incubated for 72 hours at 37° C. Across this entire range Tacrolimus appears to remain stable. The apparent elevated impurities reported in the test suspension prepared in citrate at pH 5.5 (Table 6) was likely the result of a longer incubation duration, as this test suspension was performed using a slightly different procedure than the rest of the samples with a longer incubation period and a lower concentration, 96 hours and 1 mg/g, respectively. The solubility of Tacrolimus in the supernatant was low, ranging from below the limit of quantitation to 3.2 μg/g.

Phosphate buffer, pH 7.0, was selected for further pre-formulation studies, as it is preferred to be in a pH range close to physiological pH (7.4). Several Tacrolimus suspensions were prepared in 20 mM phosphate buffer, and each containing an excipient at a concentration at or below acceptable FDA limits for ophthalmic formulations (Table 7). The stability of Tacrolimus suspensions in the presence of the excipient were measured in suspensions incubated on a rotisserie mixer for 70-72 h at 37° C. Tacrolimus suspensions appeared stable in the presence of all excipients at the tested levels.

TABLE 6
Related substances of Tacrolimus Suspensions (2 mg/g), after
72 h at 37° C., as a function of Buffer and pH Level

| | RRT's | | | | | | | | | | |
|---|---|---|---|---|---|---|---|---|---|---|---|
| Sample | | | | | | | | | | | Total |
| name | 0.65 | 0.86 | 0.96 | 1.00 | 1.18 | 1.33 | 1.37 | 1.39 | 1.44 | 1.54 | impurities |
| Citrate 5.0 | 1.65 | 1.10 | 0.77 | 95.71 | 0.58 | 0.15 | — | — | 0.06 | — | 1.56 |
| buffer | | | | | | | | | | | |
| Citrate 5.5 | 1.35 | 1.05 | 2.03 | 89.52 | 0.71 | — | 1.73 | — | 2.70 | 0.91 | 8.08 |
| buffer* | | | | | | | | | | | |
| Citrate 6.0 | 1.54 | 1.33 | 0.86 | 94.30 | 0.59 | 0.12 | 1.20 | 0.06 | — | — | 2.83 |
| buffer | | | | | | | | | | | |
| Acetate | 1.68 | 1.31 | 0.82 | 94.08 | 0.54 | 0.16 | 1.32 | 0.06 | — | — | 2.91 |
| 5.0 buffer | | | | | | | | | | | |
| Acetate | 1.66 | 1.17 | 0.79 | 94.20 | 0.62 | 0.12 | 1.35 | 0.07 | — | — | 2.95 |
| 5.5 buffer | | | | | | | | | | | |
| Acetate | 1.65 | 1.21 | 0.82 | 94.48 | 0.58 | 0.14 | 1.05 | 0.07 | — | — | 2.66 |
| 6.0 buffer | | | | | | | | | | | |
| Phosphate | 1.73 | 1.27 | 0.68 | 94.50 | 0.53 | 0.09 | — | 1.08 | 0.09 | — | 2.47 |
| 6.0 buffer | | | | | | | | | | | |
| Phosphate | 1.63 | 1.33 | 0.75 | 94.27 | 0.58 | 0.10 | 1.08 | 0.06 | — | — | 2.57 |
| 6.5 buffer | | | | | | | | | | | |
| Phosphate | 1.60 | 2.04 | 2.40 | 93.23 | 0.73 | — | — | — | — | — | 3.13 |
| 6.8 buffer | | | | | | | | | | | |
| Phosphate | 1.78 | 1.34 | 0.82 | 94.15 | 0.61 | 0.16 | 1.09 | — | — | — | 2.67 |
| 7.0 buffer | | | | | | | | | | | |
| Phosphate | 1.63 | 2.10 | 2.47 | 93.07 | 0.74 | — | — | — | — | — | 3.21 |
| 7.2 buffer | | | | | | | | | | | |
| Phosphate | 1.77 | 1.53 | 0.84 | 93.71 | 0.61 | 0.16 | 1.38 | — | — | — | 2.99 |
| 7.4 buffer | | | | | | | | | | | |

*The citrate buffer pH 5.5 was incubated for a longer duration than the other test suspensions at 96 h, instead of 72 h; this sample was prepared at 1 mg/g, instead of 2 mg/g.

TABLE 7
Excipients, function and level, evaluated in Tacrolimus suspensions

| | | FDA Max. | Study | |
|---|---|---|---|---|
| | | Permissible | Evaluation | |
| Solution | | (ophthalmic), | Load, | |
| ID | Excipient | % w/w | % w/w | Function |
| 1 | Poloxamer 188 | 0.1 | 0.10 | Dispersant, lowers |
| | | | | surface tension |
| 2 | Polysorbate 80 | 4.0 | 0.10 | Dispersant, lowers |
| | | | | surface tension |
| 3 | Glycerin | 8.8 | 1.0 | Emollient; osmolality |
| | | | | adjuster |
| 4 | Mannitol | 5.0 | 1.0 | Osmolality adjuster |
| 5 | Sodium chloride | 0.9% | 0.9 | Osmolality adjuster |
| 6 | Edetate Disodium | 0.55 | 0.10 | Chelating agent |
| 7 | Boric Acid | 5.0 | 0.10 | Stabilizing excipient |
| 8 | Carbopol 974P | 3.75 | 0.10 | Viscosifier |
| | (polycarbophil) | | | |
| 9 | HPMC | 0.6 | 0.10 | Viscosifier |
| | (Hypromellose) | | | |
| 10 | Guar Gum | 0.2 | 0.10 | Viscosifier |
| 11 | Boric acid and | See individ. | 0.1 and 1.0, | Forms complex; |
| | Mannitol | | resp | stabilizes |
| 12 | PEG 40 Stearate | 7.0% | 0.1 | Dispersant |
| | (Polyoxy1 40 stearate) | | | |

### Example 2 Micronization and Microfluidization of Tacrolimus Composition

The in vitro dissolution profiles of Tacrolimus suspensions were evaluated for different particle size distributions of Tacrolimus, to evaluate differences in the in vitro release profile as a function of the particle size distribution of Tacrolimus. The compositions of prototypes P20 and P24 were identical, but were prepared via different processes, resulting in different particle sizes of the Tacrolimus particles. Prototype P20 was prepared using micronized Tacrolimus, while P24 was prepared via a high-shear microfluidization process to result in nano-particles of Tacrolimus. The particle size distributions for P20 were 1.73 μm, 2.89 μm, 5.46 μm and span of 1.29; and for P24 were 0.23 μm, 0.85 μm, 1.63 μm and span of 1.64, for D10, D50, D90 and span, respectively.

A receptor fluid with sufficiently high solubility for Tacrolimus was required to conduct the in vitro release experiments. The solubility of Tacrolimus was evaluated in two different receptor fluid compositions containing either 1% or 5% w/w of hydroxypropyl-β-cyclodextrin (HPPCD) in a simulated tear fluid. The saturated solubility of Tacrolimus was measured at 14 μg/g, and at μg/g for 1% and 5% HPβCD receptor fluid compositions, respectively. The 5% HPβCD receptor fluid composition was chosen to conduct the in vitro release experiments, as the maximum concentration of dissolved Tacrolimus would be ˜32.2 μg/g, based on the study design.

The in vitro release experiments were performed using Float-A-Lyzer dialysis tubes (with a molecular weight cutoff of 100 kDa) containing the ˜1.3 g of suspension, which were ten placed into centrifuge tubes containing ˜39 g of receptor fluid, and placed on rotisserie incubator at 37° C. Aliquots of receptor fluid (1000 μL) were periodically removed (and replenished with fresh fluid) and were analyzed by ultra-high-pressure liquid chromatography (UHPLC).

The results for the in vitro release experiments are reported in Example 4.

The Tacrolimus suspension prepared via the microfluidization process (P24) had a higher release profile than the suspension prepared with the micronized Tacrolimus (P20), thus demonstrating that different dissolution profiles may be achieved based on the particle size distribution of the drug particles.

The corneal permeability of Tacrolimus suspensions was evaluated for prototypes of different particle size distributions of Tacrolimus, to evaluate differences in the corneal permeability as a function of the particle size distribution of Tacrolimus. The compositions of prototypes (P30 and P29) were identical but were prepared via different processes, resulting in different particle sizes of the Tacrolimus particles. Prototype P30 was prepared using micronized Tacrolimus, while P29 was prepared via a high-shear microfluidization process to result in nano-particles of Tacrolimus. The particle size distributions for P30 were 1.72 μm, 2.94 μm, 6.80 μm and span of 1.73; and for P29 were 0.28 μm, 0.78 μm, 1.68 μm and span of 1.78, for D10, D50, D90 and span, respectively.

The permeability experiments were conducted using freshly excised bovine cornea, and Franz Cell apparatuses (n=3 per suspension), with a receptor fluid of 20 mM phosphate buffer (pH 7.0) containing 5% w/w hydroxypropyl-β-cyclodextrin at 37° C. Aliquots of the receptor fluid were periodically removed (and replaced with fresh buffer), and were analyzed by ultra-high pressure liquid chromatography (UHPLC) for assay of Tacrolimus.

No Tacrolimus was detected in the receptor fluid for each suspension tested. The corneas were homogenized and extracted, and Tacrolimus was detected in the corneas. The results for the corneal permeability experiments are reported in Example 3.

Although no Tacrolimus was detected in the receptor fluid, Tacrolimus was detected in the corneas for both groups, but equivalent (albeit variable) for both groups. Tacrolimus is reported to have a log P of 3.3, indicating that it is highly lipophilic and likely permeated into the cornea epithelium (˜10% cornea thickness), but is too lipophilic to permeate into the stroma, and therefore not detected in the receptor fluid. It is concluded that the intrinsic physiochemical properties of Tacrolimus were the critical parameter in permeability, not particle size.

### Example 3-Corneal Permeability

The corneal permeability of Tacrolimus suspensions was evaluated for prototypes of different particle size distributions of Tacrolimus. The compositions of prototypes (P30 and P29) were identical but were prepared via different processes, resulting in different particle sizes of the Tacrolimus particles. Prototype P30 was prepared using micronized Tacrolimus, while P29 was prepared via a high-shear microfluidization process to result in nano-particles of Tacrolimus. The particle size distributions for P30 were 1.72 μm, 2.94 μm, 6.80 μm and span of 1.73; and for P29 were 0.28 μm, 0.78 μm, 1.68 μm and span of 1.78, for D10, D50, D90 and span, respectively.

The permeability experiments were conducted using freshly excised bovine cornea, and Franz Cell apparatuses (n=3 per suspension), with a receptor fluid of 20 mM phosphate buffer (pH 7.0) containing 5% w/w hydroxypropyl-β-cyclodextrin at 37° C. Aliquots of the receptor fluid were periodically removed (and replaced with fresh buffer), and were analyzed by UHPLC for assay of Tacrolimus. No tacrolimus was detected in the receptor fluid for each suspension tested. The corneas were homogenized and extracted. Tacrolimus was detected in the corneas for both groups, but equivalent (albeit variable) for both groups. Tacrolimus is reported to have a log P of 3.3, indicating that it is highly lipophilic and likely permeated into the cornea epithelium (˜10% cornea thickness), but is too lipophilic to permeate into the stroma, and therefore not detected in the receptor fluid. It is concluded that the intrinsic physiochemical properties of Tacrolimus was the critical parameter in permeability, not particle size.

TABLE 8
Franz Cell No. 1 (microfluidized, P29)

| | Sample | Receptor | Receptor | Conc. | Permeated |
|---|---|---|---|---|---|
| Time | Removed | Replacement | buffer | TAC | TAC |
| (h) | (g) | wt. (g) | (g) | (μg/g) | (μg) |
| 1 | 0.3 | 0.2386 | 4.9193 | 0 | 0 |
| 2 | 0.3 | 0.3093 | 4.9900 | 0 | 0 |
| 3 | 0.3 | 0.3080 | 4.9887 | 0 | 0 |
| 4 | 0.3 | 0.3212 | 5.0019 | 0 | 0 |
| 5 | 0.3 | 0.3346 | 5.0153 | 0 | 0 |
| 6 | 0.3 | 0.2849 | 4.9656 | 0 | 0 |
| 24 | 0.3 | N/A | N/A | 0 | 0 |

TABLE 9
Franz Cell No. 2 (microfluidized, P29)

| | Sample | Receptor | Receptor | Conc. | Permeated |
|---|---|---|---|---|---|
| Time | Removed | Replacement | buffer | TAC | TAC |
| (h) | (g) | wt. (g) | (g) | (μg/g) | (μg) |
| 1 | 0.3 | 0.2296 | 4.9350 | 0 | 0 |
| 2 | 0.3 | 0.3236 | 5.0290 | 0 | 0 |
| 3 | 0.3 | 0.3839 | 5.0893 | 0 | 0 |
| 4 | 0.3 | 0.2886 | 4.9940 | 0 | 0 |
| 5 | 0.3 | 0.3264 | 5.0318 | 0 | 0 |
| 6 | 0.3 | 0.2878 | 4.9932 | 0 | 0 |
| 24 | 0.3 | N/A | N/A | 0 | 0 |

TABLE 10
Franz Cell No. 3 (microfluidized, P29)

| | Sample | Receptor | Receptor | Conc. | Permeated |
|---|---|---|---|---|---|
| Time | Removed | Replacement | buffer | TAC | TAC |
| (h) | (g) | wt. (g) | (g) | (μg/g) | (μg) |
| 1 | 0.3 | 0.2617 | 4.9671 | 0 | 0 |
| 2 | 0.3 | 0.3160 | 5.0214 | 0 | 0 |
| 3 | 0.3 | 0.3599 | 5.0653 | 0 | 0 |
| 4 | 0.3 | 0.2988 | 5.0042 | 0 | 0 |
| 5 | 0.3 | 0.3105 | 5.0159 | 0 | 0 |
| 6 | 0.3 | 0.2947 | 5.0001 | 0 | 0 |
| 24 | 0.3 | N/A | N/A | 0 | 0 |

TABLE 11
Franz Cell No. 4 (micronized, 30)

| | | Receptor | | Area | | Perme- |
|---|---|---|---|---|---|---|
| | Sample | Replacement | Receptor | exposed | Conc. | ated |
| Time | Removed | wt. | buffer | to TAC | TAC | TAC |
| (h) | (g) | (g) | (g) | (mm) | (μg/g) | (μg) |
| 1 | 0.3 | 0.2147 | 4.7996 | 32 | 0 | 0 |
| 2 | 0.3 | 0.3496 | 4.9345 | 32 | 0 | 0 |
| 3 | 0.3 | 0.3709 | 4.9558 | 32 | 0 | 0 |
| 4 | 0.3 | 0.3402 | 4.9251 | 32 | 0 | 0 |
| 5 | 0.3 | 0.3005 | 4.8854 | 32 | 0 | 0 |
| 6 | 0.3 | 0.3511 | 4.9360 | 32 | 0 | 0 |
| 24 | 0.3 | N/A | N/A | 32 | 0 | 0 |

TABLE 12
Franz Cell No. 5 (micronized, 30)

| | | Receptor | | Area | | Perme- |
|---|---|---|---|---|---|---|
| | Sample | Replacement | Receptor | exposed | Conc. | ated |
| Time | Removed | wt. | buffer | to TAC | TAC | TAC |
| (h) | (g) | (g) | (g) | (mm) | (μg/g) | (μg) |
| 1 | 0.3 | 0.2107 | 4.9057 | 32 | 0 | 0 |
| 2 | 0.3 | 0.3244 | 4.9093 | 32 | 0 | 0 |
| 3 | 0.3 | 0.2643 | 4.8492 | 32 | 0 | 0 |
| 4 | 0.3 | 0.3169 | 4.9018 | 32 | 0 | 0 |
| 5 | 0.3 | 0.3031 | 4.8880 | 32 | 0 | 0 |
| 6 | 0.3 | 0.3329 | 4.9178 | 32 | 0 | 0 |
| 24 | 0.3 | N/A | N/A | 32 | 0 | 0 |

TABLE 13
Franz Cell No. 6 (micronized, 30)

| | | Receptor | | Area | | Perme- |
|---|---|---|---|---|---|---|
| | Sample | Replacement | Receptor | exposed | Conc. | ated |
| Time | Removed | wt. | buffer | to TAC | TAC | TAC |
| (h) | (g) | (g) | (g) | (mm) | (μg/g) | (μg) |
| 1 | 0.3 | 0.2014 | 4.8964 | 32 | 0 | 0 |
| 2 | 0.3 | 0.3261 | 4.9110 | 32 | 0 | 0 |
| 3 | 0.3 | 0.3277 | 4.9126 | 32 | 0 | 0 |
| 4 | 0.3 | 0.2903 | 4.8752 | 32 | 0 | 0 |
| 5 | 0.3 | 0.3324 | 4.9173 | 32 | 0 | 0 |
| 6 | 0.3 | 0.3060 | 4.8909 | 32 | 0 | 0 |
| 24 | 0.3 | N/A | N/A | 32 | 0 | 0 |

TABLE 14
Results for homogenized corneas (¼″ punched section
of each cornea), extracted for Tacrolimus at the end of the experiment

| | | Avg. | wt. of | Wt. of | | | | |
|---|---|---|---|---|---|---|---|---|
| | Franz | Thickness | cornea | diluent | TAC | TAC | TAC | Total |
| | Cell | of Cornea | sample | added | 1 | 2 | 3 | TAC |
| Formulation | No. | (mm) | (g) | (g) | AUC | AUC | AUC | AUC |
| P29 | 1 | 1.53 | 0.0053 | 1.0902 | 996 | 0 | 0 | 996 |
| Microfluidized | 2 | 1.32 | 0.0111 | 1.0880 | 6723 | 0 | 0 | 6723 |
| | 3 | 1.53 | 0.0095 | 1.0532 | 4951 | 1608 | 0 | 6559 |
| P30 | 4 | 1.53 | 0.0123 | 1.1131 | 33426 | 4400 | 0 | 37826 |
| Micronized | 5 | 1.53 | 0.0130 | 1.1196 | 3482 | 0 | 0 | 3482 |
| | 6 | 1.52 | 0.0091 | 1.1206 | 13592 | 2226 | 0 | 15818 |

TABLE 15
Results for homogenized corneas (¼″ punched section
of each cornea), extracted for Tacrolimus at the end of the experiment

| | | conc. of | | |
|---|---|---|---|---|
| | | TAC per | | TAC |
| | Conc. | weight of | Tac in | (ug)/volume |
| | observed | cornea | cornea | cornea |
| Formulation | (μg/g) | (μg/g) | (μg) | (μg/mm\hat{ }3) |
| P29 | 0.0990 | 20.35 | 0.11 | 0.002 |
| Microfluidized | 0.6683 | 66.00 | 0.73 | 0.017 |
| | 0.6520 | 72.94 | 0.69 | 0.014 |
| P30 | 3.7603 | 344.05 | 4.23 | 0.087 |
| Micronized | 0.3461 | 30.16 | 0.39 | 0.008 |
| | 1.5725 | 195.21 | 1.78 | 0.037 |

### Example 4-Tacrolimus Composition Vehicle

Several prototype formulations were evaluated using the two different procedures that had been developed for the preparation of suspensions (ref. Example 2). Prototypes varied in the choice of polymer (Guar Gum, Carbopol 974 P, and Hydroxypropyl Methylcellulose (HPMC)), and the type of dispersant (Poloxamer 188, Polyoxyl 40 Stearate, and Polysorbate 80).

The HPMC prototypes resulted in good stability, but the viscosity stability was inconsistent, and for some of the prototypes a reduction in viscosity (which generally also exhibited a loss in pH) was observed. The early HPMC prototypes were not prepared aseptically, which may be a contributing factor to the loss of viscosity. The prototypes P26 and P27, prepared using HPMC 2910 4000, both had stable pH, osmolality, PSD, assay, total impurities, and viscosities at the 5° C. condition for up to three months.

The formulation prepared using HPMC 2910 4000 cp and PEG 40 stearate was ultimately selected as the final formulation.

TABLE 16
Final composition of HPMC containing prototypes

| | P20 | P24, P26, P29 | P25, P27, P30 |
|---|---|---|---|
| | (Unprocessed) | (Microfluidized) | (Unprocessed) |
| Tacrolimus | 0.10% | 0.10% | 0.10% |
| Glycerin | 1.8% | 1.9% | 2.0% |
| Sodium Phosphate Monobasic | 0.13% | 0.13% | 0.13% |
| Monohydrate | | | |
| Dibasic Sodium Phosphate Heptahydrate | 0.28% | 0.28% | 0.28% |
| Dispersant: PEG-40 Stearate | 0.10% | 0.10% | 0.10% |
| Viscosifier: HPMC | 0.5% | 0.50% | 0.50% |
| Water | QS | QS | QS |

*P26, P27, P29, and P30 were prepared using HPMC Type 2910, 4,000 cP; the other prototypes were prepared using HPMC Type 2910, 10,000 cP

TABLE 17
One-month stability of P30, prepared using micronized
API (without additional processing) and HPMC 2910, 4000

| | | P30 Unprocessed (HPM 4000) | | | | |
|---|---|---|---|---|---|---|
| Characterization | | T0 | 2-wk, 5° C. | 2-wk, 40° C. | 1-mo, 5° C. | 1-mo, 40° C. |
| Pull Date | | 9-June | 30-Jun | 30-Jun | 15-Jul | 15-Jul |
| Osmolarity (mOsm/Kg) | | 297 | 293 | 294 | 294 | 294 |
| pH | | 7.1 | 7.0 | 6.8 | 7.1 | 6.8 |
| Viscosity (cP), 25° C. | | 20.2* | 22.0 | 10.3 | 16.6 | 7.1 |
| PSD | D10 (um) | 1.72 | 1.54 | 1.54 | 1.60 | 1.52 |
| | D50 (um) | 2.94 | 2.23 | 2.22 | 2.44 | 2.22 |
| | D90 (um) | 6.80 | 3.24 | 3.23 | 3.73 | 3.25 |
| | Span | 1.73 | 0.76 | 0.76 | 0.87 | 0.78 |

| | | | | | |
|---|---|---|---|---|---|
| Assay (mg/g) | 1.04 | 1.06 | 0.96 | 1.00 | 0.88 |
| Tot. Imp. | 0.75% | 0.68% | 1.54% | 0.73% | 1.41% |

*Viscosity measurements made on Brookfield Viscometer DVII+ CP; all other viscosity measurements made using an Anton Paar Rheometer

TABLE 18
Three-month stability for P26, prepared via microfluidization process, and contains HPMC 2910, 4000

| | | P26 Microfluidized (HPMC 4000) | | | | | | | | |
|---|---|---|---|---|---|---|---|---|---|---|
| | | To | 2-week | | 4-week | | 2-month | | 3-month | |
| Characterization | | Bulk | 5° C. | 40° C. | 5° C. | 40° C. | 5° C. | 40° C. | 5° C. | 40° C. |
| Date Pulled | | 19-May | 3-June | 3-June | 18-June | 18-June | 15-Jul | 15-Jul | 19-Aug | 19-Aug |
| Osmolarity | | 308‡ | 327 | 327 | 333 | 335 | 327 | 325 | 328 | 325 |
| (mOsm/Kg) | | | | | | | | | | |
| pH | | 7.0 | 7.1 | 7.1 | 6.9 | 7.1 | 7.1 | 7.1 | 7.0 | 7.0 |
| Viscosity (cP) | | 21.6* | 17.6 | 15.8 | 21.1* | 22.8* | 20.3 | 16.2 | 22.5 | 17.0 |
| 25° C. | | | | | | | | | | |
| PSD | D10 (um) | 0.22 | 0.36 | 0.29 | 0.37 | 0.42 | 0.27 | 0.29 | 0.47 | 0.29 |
| | D50 (um) | 0.80 | 0.74 | 0.75 | 0.76 | 0.80 | 0.79 | 0.88 | 0.81 | 0.77 |
| | D90 (um) | 1.48 | 1.02 | 1.15 | 1.57 | 1.52 | 1.45 | 2.16 | 1.07 | 1.27 |
| | Span | 1.58 | 0.89 | 1.15 | 1.57 | 1.37 | 1.53 | 2.12 | 0.74 | 1.26 |

| | | | | | | | | | |
|---|---|---|---|---|---|---|---|---|---|
| Assay (mg/g) | 1.30 | 1.22 | 1.16 | 1.06** | 1.04** | 1.02** | 0.92** | 0.96** | 0.89** |
| Tot. Imp. | | 0.80% | 1.67% | 0.21%** | 1.79%** | 0.67% | 3.51% | 0.67% | 4.80% |

‡The osmolality measurement at T0 (308 mOsm/Kg) is considered suspect, as a rise in osmolality over time has not been observed for any other prototypes
*Viscosity measurements were made on Brookfield Viscometer DVII+ CP; all other viscosity measurements made using an Anton Paar Rheometer
**Assay value reported using the optimized UHPLC sample preparation method that was later qualified (ref. Example 7)

TABLE 19
3-month stability for P27, prepared using micronized API (without additional processing) and HPMC 2910, 4000

| | | P27 Unprocessed (HPMC 4000) | | | | | | | | |
|---|---|---|---|---|---|---|---|---|---|---|
| | | To | 2-week | | 4-week | | 2-month | | 3-month | |
| Characterization | | Bulk | 5° C. | 40° C. | 5° C. | 40° C. | 5° C. | 40° C. | 5° C. | 40° C. |
| Date Pulled | | 19-May | 3-June | 3-June | 18-June | 18-June | 15-Jul | 15-Jul | 19-Aug | 19-Aug |
| Osmolarity | | 305 | 302 | 305 | 315 | 312 | 301 | 301 | 302 | 307 |
| (mOsm/Kg) | | | | | | | | | | |
| pH | | 7.0 | 7.1 | 7.1 | 7.1 | 7.1 | 7.1 | 7.1 | 7.1 | 6.8 |
| Viscosity (cP) | | 21.5* | 19.6 | 19.1 | 20.0* | 18.6* | 21.0 | 18.1 | 22.76 | 11.16 |
| 25° C. | | | | | | | | | | |
| PSD | D10 (um) | 1.65 | 1.59 | 1.68 | 1.03 | 1.42 | 1.82 | 1.80 | 1.53 | 1.49 |
| | D50 (um) | 2.70 | 2.27 | 2.44 | 2.25 | 2.81 | 2.99 | 4.93 | 2.21 | 2.18 |
| | D90 (um) | 4.91 | 3.26 | 3.64 | 4.92 | 5.56 | 5.43 | 13.46 | 3.20 | 3.21 |
| | Span | 1.21 | 0.73 | 0.80 | 1.72 | 1.48 | 1.21 | 2.36 | 0.76 | 0.79 |

| | | | | | | | | | |
|---|---|---|---|---|---|---|---|---|---|
| Assay (mg/g) | 1.32 | 1.18 | 1.15 | 1.07** | 1.03** | 1.07** | 1.08** | 0.99** | 0.90** |
| Tot. Imp. | Not Measrd. | 0.65% | 1.56% | 0.42% | 1.93% | 0.59% | 2.98% | 0.87% | 2.96% |

*Viscosity measurements were made on Brookfield Viscometer DVII+ CP; all other viscosity measurements made using an Anton Paar Rheometer
**Assay value reported using the optimized UHPLC sample preparation method that was later qualified (ref. Example 7); the earlier assay/RS values were obtained using methods under development

### Example 5-Dissolution Profiles for Tacrolimus Suspensions

The in vitro dissolution profiles for prototypes of Tacrolimus suspensions were evaluated. The compositions of the prototypes were identical, but the suspensions were prepared via different processes, resulting in different particle sizes of the Tacrolimus. Prototype P20 was prepared using micronized Tacrolimus, using API as received, that was gently mixed into the vehicle using an overhead mixer. Prototype P24 was prepared using a two-step process that involved a high shear microfluidization process of a concentrated suspension (1% w/w Tacrolimus, in a 20 mM phosphate buffer containing 0.1% w/w PEG 40 stearate, without HPMC) to reduce the Tacrolimus particles, and then was diluted using a concentrated HPMC vehicle to achieve the final composition of 0.1% Tacrolimus. The D10, D50, D90 and Span for prototype P20 were 1.73 μm, 2.89 μm, 5.46 μm and 1.29, respectively; and for P24 were 0.23 μm, 0.85 μm, 1.63 μm and 1.64, respectively.

To conduct the in vitro release experiments, it was necessary to have a receptor fluid that 20 had sufficiently high solubility for the API. The solubility of Tacrolimus was evaluated in two different receptor fluid compositions containing either 1% or 5% w/w of hydroxy propyl-β-cyclodextrin (HPβCD) in a simulated tear fluid. The solubility of Tacrolimus was 14 μg/g and 40 μg/g for 1% and 5% HPβCD receptor fluid compositions, respectively. Based on design of the in vitro release experiment, if all of the Tacrolimus available in the suspension were to dissolve (1.3 g of 1 mg/g suspension in 39 g of receptor fluid) the Tacrolimus concentration would be 32.2 μg/g. Therefore, the 5% HPβCD receptor fluid composition was chosen for the in vitro release experiments.

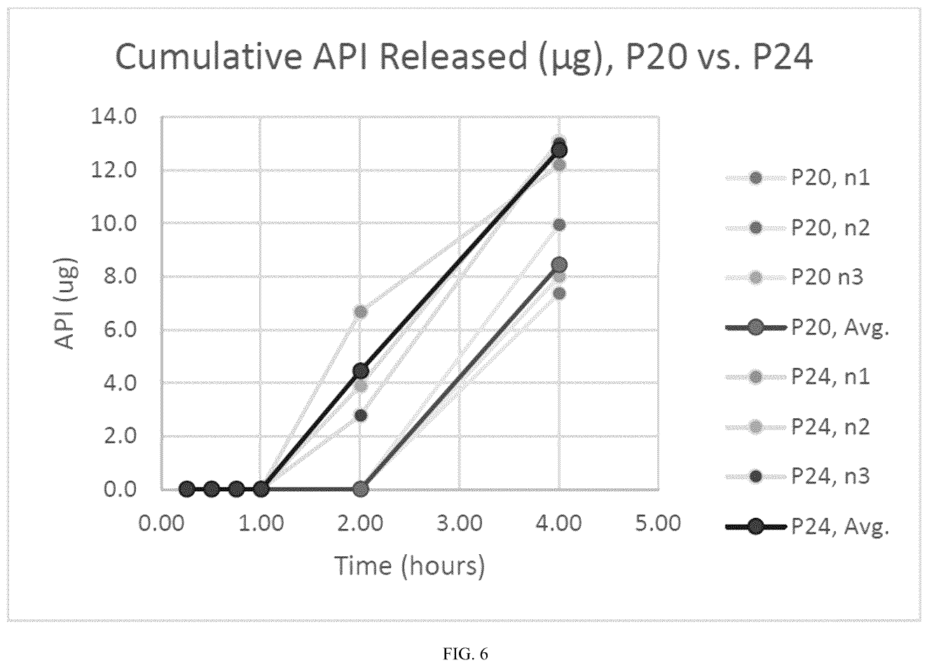

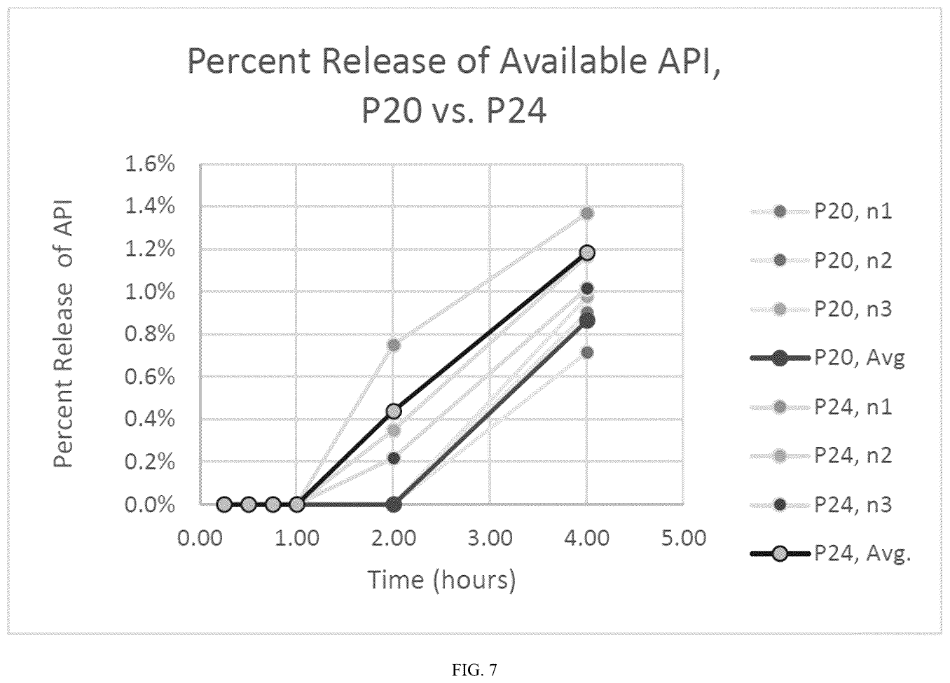

The in vitro release experiments were conducted using Float-A-Lyzer dialysis tubes with a molecular weight cutoff of 100 kDa, (each suspension in triplicate) on a rotisserie incubator, at 37° C. The presence of Tacrolimus in the initial time points (below one hour) were below the limits of quantitation. At two hours, the average amount of dissolved Tacrolimus was 4.4 μg/g (equivalent to 0.4% of available Tacrolimus) for P24, while it was below the limit of quantitation for P20 (FIG. 6). After 24 h, the P20 had released and average of 12.5%, and P24 had released an average of 8.2% of the available Tacrolimus. Thus, P24 exhibited a higher release profile than P20 in the earlier time points, but after 24 hours the amount of dissolved Tacrolimus was comparable.

The faster releaser profile of microfluidized suspension P24, as compared to P20, is likely due to the presence of Tacrolimus nanoparticles, since smaller particles are known to dissolve as a faster rate than larger particles. For an ophthalmic suspension, the faster release profile observed with the Tacrolimus suspension of nanoparticles may result in a higher bioavailability of Tacrolimus, as compared to the micronized Tacrolimus suspension.

### Example 6-Scaling the Composition

The scale-up (500 gram) preparations of the 0.1% w/w Tacrolimus suspension was attempted via two different processes. Process 1, referred to as the One-solution Process, involved the addition of micronized Tacrolimus directly to the drug vehicle; and Process 2, referred to as the Two-solution Process, by which the micronized Tacrolimus is first dispersed in a solution (Solution I), and is subsequently diluted with a viscous diluent (solution II) containing HPMC. Process 1 presented issues, and clumps of API were not readily dispersed in the viscous vehicle. Process 2 was established as the preferred process, and resulted in a drug product suspension with well dispersed API drug particles.

As part of the scale-up preparations, a filtration study was performed on the 0.5% w/w HPMC viscous solution using a PES filter (0.2 μm, Pall Supor Acropak 200, PN 12941), and the filter area required per liter was determined to be 0.0158 m².

A droplet study characterization was performed using LDPE Medidose eye dropper bottles. The average droplet weight (n=10) was 0.0323 grams (RSD=5.8%).

A settling study was conducted to determine if changes in assay value were observed in bottles that had been suspended, and allowed to rest at room temperature for up to 3 hours. Samples (at timepoints T0, 1 h, 2 h, 3 h) obtained from the bottom of individual vials (n=3 per time point), were assayed by UHPLC. No significant difference in assay measurements was observed, indicating that the suspension remains well dispersed for at least up to three hours.

TABLE 20
Composition of HPMC Vehicle used for the
One-Solution and Two-Solution Processes

| | Vehicle | |
|---|---|---|
| | (1000 g Scale) | |
| Component | Wt. % | Weight (g) |
| Viscosifier: HPMC,2910 4000 | 0.50% | 5.0 |
| Glycerin | 2.10% | 21.0 |
| Sodium Phosphate Monobasic Monohydrate | 0.11% | 1.1 |
| Dibasic Sodium Phosphate Heptahydrate | 0.33% | 3.3 |
| PEG 40 stearate | 0.10% | 1.0 |
| Water | QS | QS (968.60) |

TABLE 21
Composition of drug suspension prepared
via the One-solution Process

| | Weight |
|---|---|
| Component | Percent |
| Tacrolimus | 0.1% |
| Glycerin | 2.10% |
| Sodium Phosphate Monobasic Monohydrate | 0.11% |
| Dibasic Sodium Phosphate Heptahydrate | 0.33% |
| PEG 40 stearate | 0.10% |
| Viscosifier: HPMC,2910 4000 | 0.5% |
| Water | QS |

TABLE 22
Composition of Solution I, used in the Two-
solution process to disperse the API

| Excipients | Weight Percent |
|---|---|
| Sodium Phosphate Monobasic Monohydrate | 0.11% |
| Dibasic Sodium Phosphate Heptahydrate | 0.33% |
| PEG 40 Stearate | 0.10% |
| WFI | QS |

TABLE 23
Composition of Solution II: Viscous Diluent

| | Concentration, |
|---|---|
| Excipients | % w/w |
| HPMC 2910 (4000) | 0.50% |
| Glycerin | 2.35% |
| Sodium Phosphate Monobasic Monohydrate | 0.11% |
| Dibasic Sodium Phosphate Heptahydrate | 0.33% |
| Dispersant: PEG 40 Stearate | 0.10% |
| WFI | QS |

TABLE 24
Composition of drug suspension prepared
via the Two-solution Process

| | Weight |
|---|---|
| Components | percent |
| Tacrolimus | 0.10% |
| Glycerin | 2.12% |
| Sodium Phosphate Monobasic Monohydrate | 0.11% |
| Dibasic Sodium Phosphate Heptahydrate | 0.33% |
| Dispersant: PEG 40 Stearate | 0.10% |
| Viscosifier: HPMC 2910 (4000) | 0.45% |
| WFI | QS |

TABLE 25
Characterization results obtained for scaled-up batch (lot
CN2.1717.BR.01.20210723), prepared via the Two-solution process
Characterization

| Osmolarity (mOsm/Kg) | | 300 |
|---|---|---|
| pH | | 7.10 |
| Viscosity (cP), 25° C., SR: 50 1/s | | 23.4 |
| PSD | D10 (μm) | 1.72 |
| | D50 (μm) | 2.67 |
| | D90 (μm) | 4.44 |
| | Span | 1.02 |

| | |
|---|---|
| Assay (mg/g) | 1.03 |
| Total Impurity (%) | 0.62% |

TABLE 26
Results from the settling study of Lot CN2.1717.BR.01.20201723

| | | | | | | | | | | Total |
|---|---|---|---|---|---|---|---|---|---|---|
| | | | | | | | | | | [API] |
| | Drug | | | | TAC1 | TAC2 | TAC3 | | measrd | in |
| Sample | Product | 20% | Diluent | IPA | AUC, | AUC, | AUC, | | [API] | sample |
| ID | (g) | NaCl | (g) | (g) | RT 9.0 | RT 7.7 | RT 5.9 | Sum AUC | mg/g | (mg/g) |
| T0, n1 | 0.2136 | 0.0431 | 0.7319 | 0.0388 | 1,859,905 | 18,691 | 35,964 | 1,914,560 | 0.1922 | 0.92 |
| T0, n2 | 0.2146 | 0.0461 | 0.7250 | 0.0396 | 1,861,878 | 18,667 | 37,312 | 1,917,857 | 0.1925 | 0.92 |
| 1 h, n1 | 0.2138 | 0.0527 | 0.7163 | 0.0410 | 1,959,938 | 19,499 | 38,174 | 2,017,611 | 0.2025 | 0.97 |
| 1 h, n2 | 0.2147 | 0.0434 | 0.7131 | 0.0402 | 1,868,313 | 19,370 | 36,554 | 1,924,237 | 0.1931 | 0.91 |
| 2 h, n1 | 0.2130 | 0.0456 | 0.7138 | 0.0420 | 1,883,183 | 19,229 | 37,679 | 1,940,091 | 0.1947 | 0.93 |
| 2 h, n2 | 0.2163 | 0.0399 | 0.7135 | 0.0415 | 1,883,277 | 19,959 | 37,200 | 1,940,436 | 0.1948 | 0.91 |
| 3 h, n1 | 0.2138 | 0.0443 | 0.7131 | 0.0403 | 1,868,921 | 19,778 | 36,948 | 1,925,647 | 0.1933 | 0.91 |
| 3 h, n2 | 0.2124 | 0.0449 | 0.7145 | 0.0414 | 1,851,132 | 19,652 | 37,033 | 1,907,817 | 0.1915 | 0.91 |

Note:
The contents from the study designated bottles were combined, and fresh bottles were refilled, due to a technical error; the low assay results (as compared to the T0 results shown in Table X) is likely due to losses occurring in the collecting and rebottling of these test samples.

TABLE 27
Summary of Analysis of Variance of Settling
Study (ANOVA: Single Factor)

| Groups | Count | Sum | Average | Variance |
|---|---|---|---|---|
| T0 | 2 | 1.84 | 0.92 | 0 |
| 1 h | 2 | 1.88 | 0.94 | 0.0018 |
| 2 h | 2 | 1.84 | 0.92 | 0.0002 |
| 3 h | 2 | 1.82 | 0.91 | 0 |

| Source of Variation | SS | df | MS | F | P-value | F crit |
|---|---|---|---|---|---|---|
| Between Groups | 0.00095 | 3 | 0.000316667 | 0.63 | 0.63 | 6.59 |
| Within Groups | 0.002 | 4 | 0.0005 | | | |
| Total | 0.00295 | 7 | | | | |

### Example 7 Tacrolimus Ophthalmic Suspension Drug Product

An ultra-high-performance chromatography (UHPLC) method to measure the concentration and related substances of Tacrolimus (TAC) present in a 0.1% w/w Tacrolimus drug suspension was qualified. A critical aspect of the method qualification was the test sample preparation of the drug product, which includes multiple steps required to precipitate the hydroxypropyl methylcellulose (HPMC) polymer from the drug suspension prior to UHPLC analysis. The precipitation of the HPMC presents a challenge as the polymer tends to remain hydrated. As part of the sample preparation, a salt solution was added, which facilitated the removal of the water from the HPMC, and resulted in two liquid phases. By the addition of isopropanol, the two liquid phases formed one liquid phase layer, while the polymer remained undissolved. However, given time, the polymer would tend to hydrate in the liquid solution after the addition of the isopropanol. Thus, the removal of the supernatant before the polymer becomes hydrated, is time-sensitive, and a critical step for the test sample preparation.

Forced degradation of the drug product by thermal, acidic, basic, oxidative and UV stress conditions was performed. Forced degradation for the thermal, acidic and oxidative conditions were performed at 40° C., as it was anticipated that the drug would be stable under these conditions, based on its structure. UV and basic conditions were performed at ambient conditions. The drug product is most susceptible to basic stress conditions, and to a lesser extent is susceptible to UV and oxidative conditions. A summary of all the degradation peaks as a function of the stress condition can be found in Table 28.

TABLE 28
Summary of degradation impurities for Tacrolimus
as a function of the stress condition

| | | Relative Retentions Times (RRT) for |
|---|---|---|
| | | Degradants of Tacrolimus as a function |
| | Exposure | of stress condition |
| | Time | Area % |
| Thermal | 72 h (40° C.) | 0.43 (0.96%) |
| UV | 72 h (ambient) | 0.38 (0.36%) |
| | | 0.45 (0.88%) |
| | | 0.53 (3.07%) |
| | | 0.58 (0.40%) |
| | | 1.03 (1.30%) |
| | | 1.10 (0.94%) |
| | | 1.13 (2.90%) |
| | | 1.17 (5.21%) |
| | | 1.21 (13.81%) |
| Peroxide | 48 h (40° C.) | 0.43 (2.03%) |
| | | 1.18 (0.17%) |
| | | 1.20 (0.05%) |
| | | 1.25 (0.35%) |
| | | 1.27 (0.16%) |
| | | 1.29 (0.10%) |
| | | 1.33 (0.06%) |
| | | 1.44 (0.08%) |
| Acid | 72h (40° C.) | 0.52 (0.47%) |
| | | 1.32 (0.09%) |
| | | 1.36 (0.07%) |
| Base | 5 min | 0.39 (1.52%) |
| | (ambient) | 0.52 (27.62%) |
| | | 0.79 (0.97%) |
| | | 1.28 (0.20%) |
| | | 1.32 (3.02%) |
| | | 1.35 (2.37%) |

The HPLC method was qualified with respect to linearity, quantitation limit (LOQ, 0.125 ag/g, and is 0.05% of the test sample target of 0.25 mg/g), system precision, method precision, and intermediate precision, system suitability, peak purity, specificity (including the stressed degradants), solution stability (24 h in sample tray at 15° C.), and accuracy. No matrix effect was found. This method meets all acceptance criteria, and is thus qualified for the assay of Tacrolimus and its related substances in the 0.1% w/w (1 mg/g) Tacrolimus ophthalmic drug suspension.

TABLE 29
Composition of 0.1% Tacrolimus Ophthalmic Suspension

| | Limit: FDA | | |
|---|---|---|---|
| | Inactive | | |
| | Ingredient for | | |
| | Ophthalmic Eye | Conc, % | |
| Excipients | Solutions | w/w | Justification |
| Tacrolimus | — | 0.10% | Active |
| Glycerin | 2.5% w/v | 2.12% | Tonicity |
| | | | Adjustment |
| Sodium Phosphate, | 1.3% w/v | 0.11% | Buffer |
| Monobasic, Monohydrate | | | Component |
| Sodium Phosphate, | 2.15% w/v | 0.33% | Buffer |
| Dibasic, Heptahydrate | | | Component |
| Dispersant: Polyoxy1 40 | 7.0% w/w | 0.10% | Particle |
| Stearate | | | Stabilizer |
| Viscofier: HPMC Type | 0.6% w/v | 0.45% | Viscofier |
| 2910 (4000 cP) | | | and particle |
| | | | stabilizer |
| WFI | — | QS | |

TABLE 30
Composition of Placebo

| | Concentration, |
|---|---|
| Excipients | % w/w |
| Glycerin | 2.12% |
| Sodium Phosphate, Monobasic, Monohydrate | 0.11% |
| Sodium Phosphate, Dibasic, Heptahydrate | 0.33% |
| Dispersant: PEG 40 Stearate | 0.10% |
| Viscofier: HPMC Type 2910 (4000 cP) | 0.45% |
| WFI | QS |

TABLE 31
Characteristics of Representative Formulations of 1 mg/g Tacrolimus Suspensions

| | Lot: | | |
|---|---|---|---|
| TEST | CN2.1717.BR.01.20210723 | Prototype P30 | Prototype P27 |
| API Source | Biocon, Lot BS20006423 | Biocon, Lot BS20006423 | Biocon, Lot BS20006423 |
| Appearance of | Opaque or slightly opaque, | Opaque or slightly | Opaque or slightly |
| Suspension | white; particles freely | opaque, white; particles | opaque, white; |
| | suspend after shaking | freely suspend after | particles |
| | | shaking | freely suspend after |
| | | | shaking |
| Assay (mg/g) | 1.03 | 1.04 | 1.18 |
| pH | 7.1 | 7.1 | 7.1 |
| Osmolality (mOsm/Kg) | 300 | 297 | 302 |
| Particle Size Distribution | D10: 1.72 μm | D10: 1.72 μm | D10: 1.59 μm |
| of Suspension | D50: 2.67 μm | D50: 2.94 μm | D50: 2.27 μm |
| | D90: 4.44 μm | D90: 6.80 μm | D90: 3.26 μm |
| | Span: 1.02 | Span: 1.73 | Span: 0.73 |
| Viscosity (cP) @ 25° C. | 23.4 | 20.2* | 19.6* |
| 50 1/s shear rate | | | |
| Related Substances | RRT Impurities ^{‡} | Tot. Imp .: 0.75% | RRT Impurities |
| | 0.64 (TAC 3) 1.73% | | 0.65 (TAC 3) 2.22% |
| | 0.85 (TAC 2) 0.80% | | 0.85 (TAC 2) 1.84% |
| | 0.95 (ASC) 0.52% | | 0.95 (ASC) 0.652% |
| | 1.00 (TAC 1) 96.85% | | 1.00 (TAC 1) 95.29% |
| | 1.41 0.10% | | |
| | Tot. Imp .: 0.62% | | Tot. Imp .: 0.62% |

* Viscosity measurements obtained using Brookfield cone and plate Viscometer DVII+; other viscosity measurements were made using an Anton Paar Rheometer Model MCR 92
^{‡} Only peaks other than TAC1, TAC2, and TAC3 are counted as impurities

### Example 8-Micronized Tacrolimus Suspension Stability

The chemical and physical stability of a 0.1% w/w (1 mg/g) micronized Tacrolimus drug suspension, Lot CN2.1717.BR.01.20210723, was evaluated per ICH stability guidelines of new drug products.

After three months on stability, the formulation was found to be stable for assay, viscosity, osmolality, pH, and PSD at the 5° C. and 25° C./60% RH. At 40° C./75% RH significant decreases in pH (from 7.10 to 6.68) and viscosity (from 23.4 to 6.7 cP) were observed. The initial total impurities at TO (0.62%) increased across all temperature conditions to 1.34%, 1.55%, and 2.72%, at 5° C., 25° C., and 40° C., respectively. Due to this trend it is recommended that long term storage of the product be at 5° C. The formulation shows instability at elevated temperature, particularly at 40° C.

TABLE 32
Composition of 1 mg/g Micronized Tacrolimus Drug Suspensions,
Lot # CN2.1717.BR.01.20210723

| | Conc, % |
|---|---|
| Excipients | w/w |
| Tacrolimus | 0.10% |
| Glycerin | 2.12% |
| Sodium Phosphate, Monobasic, Monohydrate | 0.11% |
| Sodium Phosphate, Dibasic, Heptahydrate | 0.33% |
| Dispersant: Polyoxy1 40 Stearate | 0.10% |
| Viscofier: HPMC Type 2910 (4000 cP) | 0.45% |
| WFI | QS |

TABLE 33
1 mg/g Tacrolimus Drug Suspension Lot CN2.1717.BR.01.20210723, 5° C.

| Characterization | | Specification | T0 | 6 weeks | 3 Months | 6 Months |
|---|---|---|---|---|---|---|
| Appearance | | Opaque or | Conforms | Conforms | Conforms | |
| | | slightly opaque, | | | | |
| | | white; particles | | | | |
| | | freely suspend | | | | |
| | | after shaking | | | | |
| Assay | | 1.0 mg/g | 1.03 | 1.00 | 1.01 | |
| | | (0.9-1.1) | | | | |
| Related | | <1.0% Tot. | Tot. Imp. 0.62%* | 0.83%* | 1.34%* | %* |
| Substances | | Imp. Or TBD | RRT % Area | RRT % Area | RRT % Area | RRT % Area |
| | | by CN2 | 0.65 TAC3 1.73% | 0.65 TAC3 2.12% | 0.65 TAC3 1.85% | 0.65 TAC3 |
| | | | 0.85 TAC2 0.80% | 0.71 0.15% | 0.70 0.41% | 0.85 TAC2 |
| | | | 0.95 ASC 0.52% | 0.85 TAC2 0.90% | 0.85 TAC2 0.80% | 0.95 ASC |
| | | | 1.0 TAC1 96.86% | 0.95 ASC 0.54% | 0.95 ASC 0.83% | 1.0 TAC1 |
| | | | 1.41 0.10% | 1.0 TAC1 96.16% | 1.0 TAC1 96.01% | |
| | | | | 1.44 0.14% | 1.42 0.10% | |
| pH | | Target: 7.0 | 7.10 | 7.07 | 7.11 | |
| | | (6.8-7.2) | | | | |
| Osmolality | | Target: 295 | 300 | 298 | 299 | |
| (mOsm/kg) | | (280-310) | | | | |
| Average, n = 2 | | | | | | |
| Viscosity (cP) | | Target: 25 cP | 23.4 | 23.3 | 20.5 | |
| @ 25° C., shear | | (15-35) | | | | |
| rate 50 s⁻¹ | | | | | | |
| PSD | D10 (μm) | N/A | 1.72 | 1.53 | 1.81 | |
| | D50 (μm) | 1.5-3.5 | 2.67 | 2.22 | 2.90 | |
| | D90 (μm) | 3.0-8.0 | 4.44 | 3.22 | 5.07 | |
| | Span | N/A | 1.02 | 0.77 | 1.12 | |

*Only peaks other than TAC1, TAC2, and TAC3 are counted as impurities

TABLE 34
1 mg/g Tacrolimus Drug Suspension Lot CN2.1717.BR.01.20210723, 25° C./60% RH

| Characterization | | Specification | T0 | 6 weeks | 3 Months | 6 Months |
|---|---|---|---|---|---|---|
| Appearance | | Opaque or | Conforms | Conforms | Conforms | |
| | | slightly opaque, | | | | |
| | | white; particles | | | | |
| | | freely suspend | | | | |
| | | after shaking | | | | |
| Assay | | 1.0 mg/g | 1.03 | 0.97 | 1.00 | |
| | | (0.9-1.1) | | | | |
| Related | | <1.0% Tot. | Tot. Imp. 0.62%* | 0.73%* | 1.55%* | %* |
| Substances | | Imp. Or TBD | RRT % Area | RRT % Area | RRT % Area | RRT % Area |
| | | by CN2 | 0.65 TAC3 1.73% | 0.65 TAC3 2.08% | 0.53 0.63% | 0.65 TAC3 |
| | | | 0.85 TAC2 0.80% | 0.71 0.23% | 0.65 TAC3 1.70% | 0.85 TAC2 |
| | | | 0.95 ASC 0.52% | 0.85 TAC2 0.77% | 0.85 TAC2 0.77% | 0.95 ASC |
| | | | 1.0 TAC1 96.86% | 0.95 ASC 0.50% | 0.95 ASC 0.69% | 1.0 TAC1 |
| | | | | 1.41 0.10% | 1.0 TAC1 96.42% | 1.0 TAC1 95.99% |
| | | | | | | 1.32 0.07% |
| | | | | | | 1.36 0.05% |
| | | | | | | 1.42 0.11% |
| pH | | Target: 7.0 | 7.10 | 7.06 | 7.09 | |
| | | (6.8-7.2) | | | | |
| Osmolality | | Target: 295 | 300 | 302 | 301 | |
| (mOsm/kg) | | (280-310) | | | | |
| Average, n = 2 | | | | | | |
| Viscosity (cP) | | Target: 25 cP | 23.4 | 22.9 | 26.5 | |
| @ 25° C., shear | | (15-35) | | | | |
| rate 50 s⁻¹ | | | | | | |
| PSD | D10 (μm) | N/A | 1.72 | 1.49 | 1.49 | |
| | D50 (μm) | 1.5-3.5 | 2.67 | 2.18 | 2.86 | |
| | D90 (μm) | 3.0-8.0 | 4.44 | 3.20 | 5.52 | |
| | Span | N/A | 1.02 | 0.79 | 1.40 | |

*Only peaks other than TAC1, TAC2, and TAC3 are counted as impurities

TABLE 35
1 mg/g Tacrolimus Drug Suspension Lot CN2.1717.BR.01.20210723, 40° C./75% RH

| Characterization | | Specification | T0 | 6 weeks | 3 Months | 6 Months |
|---|---|---|---|---|---|---|
| Appearance | | Opaque or | Conforms | Conforms | conforms | |
| | | slightly opaque, | | | | |
| | | white; particles | | | | |
| | | freely suspend | | | | |
| | | after shaking | | | | |
| Assay | | 1.0 mg/g | 1.03 | 0.98 | 0.90 | |
| | | (0.9-1.1) | | | | |
| Related | | <1.0% Tot. | Tot. Imp. 0.62%* | 2.50%* | 2.72% * | %* |
| Substances | | Imp. Or TBD | RRT % Area | RRT % Area | RRT % Area | RRT % Area |
| | | by CN2 | 0.65 TAC3 1.73% | 0.52 1.43% | 0.40 0.40% | 0.65 TAC3 |
| | | | 0.85 TAC2 0.80% | 0.65TAC3 2.33% | 0.53 1.38% | 0.85 TAC2 |
| | | | 0.95 ASC 0.52% | 0.85 TAC2 0.78% | 0.65 TAC3 1.79% | 0.95 ASC |
| | | | 1.0 TAC1 96.86% | 0.95 ASC 0.48% | 0.85 TAC2 0.67% | 1.0 TAC1 |
| | | | 1.41 0.10% | 1.0 TAC1 94.38% | 0.95 ASC 0.57% | |
| | | | | 1.20 0.24% | 1.0 TAC1 94.82% | |
| | | | | 1.35 0.16% | 1.16 0.16% | |
| | | | | 1.36 0.09% | 1.32 0.14% | |
| | | | | 1.38 0.10% | 1.36 0.08% | |
| pH | | Target: 7.0 | 7.10 | 7.02 | 6.68 | |
| | | (6.8-7.2) | | | | |
| Osmolality | | Target: 295 | 300 | 300 | 299 | |
| (mOsm/kg) | | (280-310) | | | | |
| Average, n = 2 | | | | | | |
| Viscosity (cP) | | Target: 25 cP | 23.4 | 17.7 | 6.7 | |
| @ 25° C., shear | | (15-35) | | | | |
| rate 50 s⁻¹ | | | | | | |
| PSD | D10 (μm) | N/A | 1.72 | 1.61 | 1.50 | |
| | D50 (μm) | 1.5-3.5 | 2.67 | 2.26 | 2.21 | |
| | D90 (μm) | 3.0-8.0 | 4.44 | 3.24 | 3.30 | |
| | Span | N/A | 1.02 | 0.72 | 0.81 | |

*Only peaks other than TAC1, TAC2, and TAC3 are counted as impurities

The composition can be irradiated at a dose ranging from about 1 kilo-Gray (kGy) to about 100 kGy, preferably about 10 kGy to about 75 kGy, most preferably about 15 kGy to about 40 kGy (Table 36).

TABLE 36
Irradiation

| | | | Total [Tac] in | | |
|---|---|---|---|---|---|
| | Appearance | | sample | | |
| | (Type I Glass | Actual [TAC] | measured | | |
| Sample ID | vial) | (mg/g) | (mg/g) | % recovery | % impurities |
| Control solid | Clear | 0.9988 | 0.99 | 98.91% | 0.56% |
| 15 kGy solid | Amber | 0.9994 | 0.96 | 95.94% | 0.82% |
| 25 kGy solid | Amber | 0.9970 | 0.92 | 92.55% | 0.88% |
| 40 kGy solid | Amber | 0.9951 | 0.91 | 91.92% | 0.91% |

### Example 9-Droplet Dose

As part of the formulation development, the average droplet weight was previously measured as 32 mg per droplet (n=1, 5.8% RSD; ref. Example 6). This evaluation was conducted using the same type of eye dropper bottle. The current evaluation resulted in a slightly larger average droplet weight of 40.4 mg (n=10, 9.7% RSD). The droplet size and weight are likely to vary as a function of the opening of the bottle tip. The average measured concentration of Tacrolimus per droplet was 0.97 mg/g (n=10, 2.25% RSD), and the average dose of Tacrolimus per droplet was determined as 39.2 mg (n=10, 11.1% RSD). Overall, the droplet weight and Tacrolimus dose per droplet is considered to be consistent and uniform.

TABLE 37
Dose per droplet results

| Drop # | Droplet wt. (g) | Observed [Tac] (mg/g) | Dose per drop (g) |
|---|---|---|---|
| 1 | 0.0375 | 0.9713 | 0.036 |
| 2 | 0.0422 | 0.9557 | 0.040 |
| 3 | 0.0341 | 0.9228 | 0.031 |
| 4 | 0.0436 | 0.9916 | 0.043 |
| 5 | 0.0369 | 0.9423 | 0.035 |
| 6 | 0.0385 | 0.9778 | 0.038 |
| 7 | 0.0430 | 0.9772 | 0.042 |
| 8 | 0.0474 | 0.9750 | 0.046 |
| 9 | 0.0388 | 0.9881 | 0.038 |
| 10 | 0.0420 | 0.9825 | 0.041 |
| Avg. | 0.0404 | 0.9684 | 0.039 |
| St. dev. | 0.0039 | 0.0218 | 0.0043 |
| RSD | 9.70% | 2.25% | 11.07% |

TABLE 38
Droplet weight during the Scale-up Process

| No. | Droplet Weight (g) |
|---|---|
| 1 | 0.0309 |
| 2 | 0.0316 |
| 3 | 0.0319 |
| 4 | 0.0310 |
| 5 | 0.0306 |
| 6 | 0.0342 |
| 7 | 0.0337 |
| 8 | 0.0362 |
| 9 | 0.0322 |
| 10 | 0.0304 |
| Ave. | 0.0323 |
| Stnd. Dev. | 0.0019 |
| RSD | 5.8% |

TABLE 39
Composition Specification OSD (APPLICATOR 2.4 mm)

| Item | Description | Specification | | Unit | Test method |
|---|---|---|---|---|---|
| Dose volume | Dose after | Filing volume | 35 ± 10 | μl | Manual |
| | priming in 180° | 10 ml | | | actuation |
| | upside down | | | | Test - Fluid |
| | position | | | | (deionized |
| | | | | | water. 0.9% |
| | | | | | NaCl) |
| Actuation | Force to actuate | Filling | ≤25 | N | Actuation |
| force | the OSD | according to | | | by |
| | | nominal bottle | | | appropriate |
| | | volume | | | actuation |
| | | | | | equipment |
| | | | | | Test - Fluid |
| | | | | | (deionized |
| | | | | | water. 0,9% |
| | | | | | NaCl) |
| Leakage | Tightness of | 15 min absolute | No visible | — | Exsiccator |
| | the OSD | pressure 750 | leakage | | |
| | system with | mbar | | | |
| | correctly | | | | |
| | assembled | | | | |
| | container and | | | | |
| | originally | | | | |
| | sealed devices | | | | |
| | with tamper | | | | |
| | evidence ring | | | | |
| dropping | Visual control | No jet, no suck back, | | — | Manual |
| | of the drop | controlled dropping | | | actuation |
| | formation in | | | | Test - Fluid |
| | 180° upside | | | | (deionized |
| | down position | | | | water. 0.9% |
| | | | | | NaCl) |

### Example 10 Tacrolimus Composition Surface Tension and Contact Angle Measurements

The Theta Lite Optical Tensiometer, Pendant drop Method, 5 μL sample size was used to measure the surface tension of the tacrolimus compositions.

TABLE 40
Surface Tension

| | Surface Tension |
|---|---|
| Solutions/Suspensions | (mN/m) |
| CN2 0.1% w/w (1 mg/g) Tacrolimus Suspension | 46.6 |
| Water | 72 |
| Normal Tear Fluid | 40-50 |
| Azopt Brinzolamide Suspension | 34.45-35.04 |

The Theta Lite Optical Tensiometer, Dynamic Contact Angle Measurement, 20 μL sample size was used to measure the contact angle of the tacrolimus compositions of parafilm of 73.50 and 73.20. The tacrolimus composition has better wettability on the hydrophobic surface of parafilm when compared to water (97.90 and 99.90).

It should be understood that the examples and embodiments described herein are for illustrative purposes only and that various modifications or changes in light thereof will be suggested to persons skilled in the art and are to be included within the spirit and purview of this application and the scope of the appended claims. In addition, any elements or limitations of any invention or embodiment thereof disclosed herein can be combined with any and/or all other elements or limitations (individually or in any combination) or any other invention or embodiment thereof disclosed herein, and all such combinations are contemplated with the scope of the invention without limitation thereto.

1. A composition comprising tacrolimus according to Formula (I), Formula (II), Formula (III), or any combination thereof, a carrier, a viscosifier, a dispersant, an emollient, Hydroxypropyl-β-cyclodextrin, and a buffer, and excluding a preservative, wherein the tacrolimus is provided as a particle with a size of less than 5 μm, wherein the tacrolimus is sterilized by gamma irradiation, wherein the carrier comprises squalene at a concentration of about 1 to about 10% v/v, wherein the composition is formulated to be dispensed from a dropper or drip chamber in a single drop, wherein the single drop comprises an amount of tacrolimus of about 0.5 mg to about 0.8 mg, wherein the composition has a viscosity of 6.7 centipoise (cP) to about 25 cP, wherein the particle size of the composition is about 0.2 μm to about 0.9 μm, and wherein:

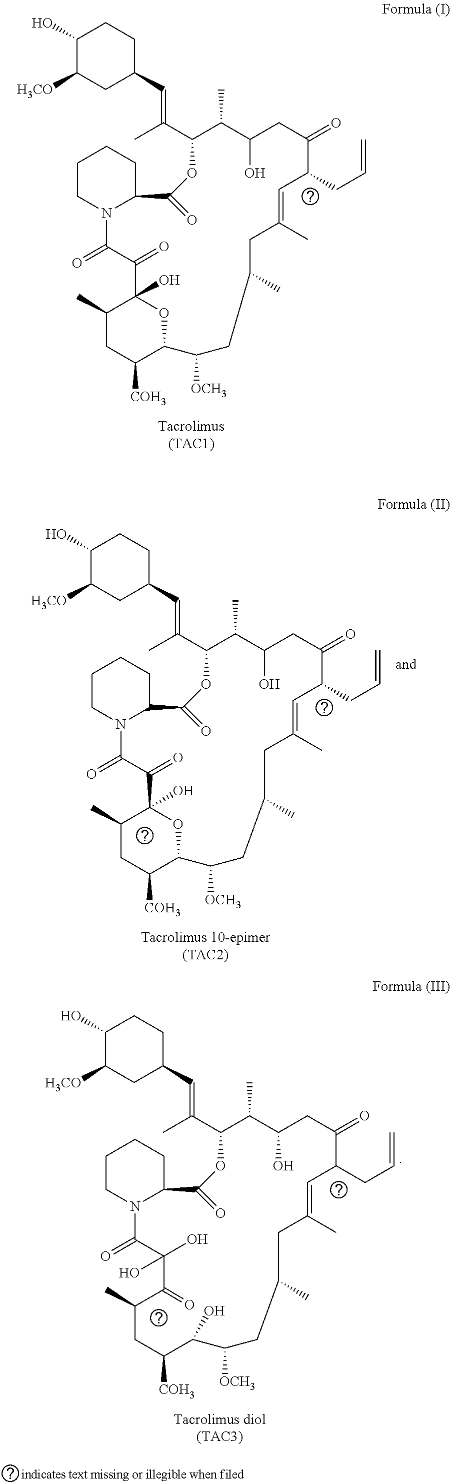

2. The composition of claim 1, wherein the emollient comprises about 0.1% to about 10% of the composition and is glycerin.

3. The composition of claim 1, wherein the composition has a pH of about 7 to about 8.

4. (canceled)

5. The composition of claim 1, wherein the dispersant comprises about 0.01% to about 10% of the composition and is polyethylene glycol (PEG) 40 stearate.

6. The composition of claim 1, wherein the viscosifier comprises about 0.01% to about 10% of the composition and is hydroxypropyl methylcellulose (HPMC).

7. The composition of claim 1, wherein the buffer comprises about 0.01% to about 10% of the composition and is phosphate monobasic monohydrate, dibasic sodium phosphate heptahydrate, or a combination thereof.

8-9. (canceled)

10. The composition of claim 1, wherein the contact angle of the compositions are about 68.0° to about 79.0°.

11. A method of treating ocular itchiness, ocular redness, or a combination thereof due to allergic conjunctivitis in a subject, the method comprising administering a composition according to claim 1 to the subject.

12. The method of claim 11, wherein the composition is administered to the eye of the subject.

13. The method of claim 11, wherein the composition is administered 1, 2, 3, or 4-times per day.

14. The method of claim 11, wherein the composition is administered for about 7 days, about 14 days, about 21 days, about 28 days, about 30 days, about 2 months, about 3 months, about 6 months, or about 1 year.

15. The method of claim 14, wherein at least one drop, at least two drops, or at least three drops are administered to the eye for each dose of the composition administered.

16. The method of claim 15, wherein the at least one drop comprises a dose of tacrolimus of about 30 mg to about 50 mg.

17. The method of claim 11, wherein the composition is administered by a multidose delivery system comprising a tip seal and filter.

18. The method of claim 17, wherein the tip seal closes an opening of a tip of the multidose delivery system immediately when releasing the pressure.

19. The method of claim 17, wherein the filter filters air through a membrane to reduce or eliminate impurities or contaminants from air required to compensate the volume loss in the delivery system after actuation of the delivery system.

20. A method for synthesizing a product of interest, the method comprising:
mixing tacrolimus into Solution I by overhead mixing; and
diluting the tacrolimus and solution I suspension with solution II,
wherein solution I comprises sodium phosphate monobasic monohydrate, dibasic sodium phosphate heptahydrate, and a dispersant; and solution II comprises a viscosifier, glycerin, sodium phosphate monobasic monohydrate, dibasic sodium phosphate heptahydrate, and a dispersant, and
wherein the product of interest has a pH of about 7 to about 8 and a concentration of 0.1% tacrolimus and excludes a preservative.

21. The method of claim 20, wherein the concentration of tacrolimus mixed into solution I is about 4 mg/g.

22. The method of claim 20, wherein the tacrolimus suspension in solution I is diluted at a 1:3 ratio relative to solution II.

23. The method of claim 20, wherein the dispersant is polyoxyl 40 stearate and the viscosifier is hydroxypropyl cellulose.

24. The method of claim 20, wherein the product of interest is stable at about 5° C. to about 40° C. for about 3 months.

25. A method for synthesizing a product of interest, the method comprising:
mixing tacrolimus into solution I by overhead mixing;
microfluidizing the tacrolimus and solution I mixture with compressed air; and
diluting the tacrolimus and solution I suspension with solution II or solution II and additional solution I,
wherein solution I comprises sodium phosphate monobasic monohydrate, dibasic sodium phosphate heptahydrate, and a dispersant; and solution II comprises a viscosifier, glycerin, sodium phosphate monobasic monohydrate, dibasic sodium phosphate heptahydrate, and a dispersant,
wherein the product of interest has a pH of about 7 to about 8 and a concentration of 0.1% tacrolimus and excludes a preservative, and
wherein microfluidizing comprises 20 passes of the tacrolimus suspension in solution I through a microfluidization instrument.

26. The method of claim 25, wherein the concentration of tacrolimus mixed into solution I is about 10 mg/g.

27. The method of claim 25, wherein the tacrolimus suspension in solution I is diluted at a 1:9 ratio relative to solution II or solution II and the additional solution I.

28. The method of claim 25, wherein the dispersant is polyoxyl 40 stearate and the viscosifier is hydroxypropyl cellulose.

29. The method of claim 25, wherein the microfluidizing uses a Z-channel configuration.

30. The method of claim 25, wherein the product of interest is stable at about 5° C. to about 40° C. for about 3 months.
